(19) United States
(12) Patent Application Publication
(10) Pub. No.: US 2023/0184771 A1
(43) Pub. Date: Jun. 15, 2023

(54) METHODS FOR TREATING BLADDER CANCER

(71) Applicant: Board of Regents, The University of Texas System, Austin, TX (US)

(72) Inventors: Padmanee Sharma, Houston, TX (US); James Allison, Houston, TX (US); Sangeeta Goswami, Houston, TX (US); Hao Zhao, Houston, TX (US)

(73) Assignee: Board of Regents, The University of Texas System, Austin, TX (US)

(21) Appl. No.: 17/996,828

(22) PCT Filed: Apr. 20, 2021
(86) PCT No.: PCT/US2021/028253

§ 371 Date: Oct. 21, 2022

Related U.S. Application Data
(60) Provisional application No. 63/013,026, filed on Apr. 21, 2020.
(60) Provisional application No. 63/159,263, filed on Mar. 10, 2021.

(51) Int. Cl. G01N 33/574; A61P 35/00; A61P 13/10; C07K 16/28; A61K 45/06; A61K 39/395

(52) U.S. Cl. CPC G01N 33/57407; A61P 35/00; A61P 13/10; C07K 16/2818; A61K 45/06; A61K 39/39558; G01N 2333/7158; A61K 2039/505

(57) ABSTRACT

The current disclosure provides for novel therapeutic methods by identifying cancer patient populations that may be treated effectively by immunotherapies. Accordingly, aspects of the disclosure relate to a method of treating cancer in a subject comprising administering to the subject immune checkpoint blockade (ICB) therapy after the subject has been determined to have increased expression of CXCL13 in a biological sample from the subject.

### BACKGROUND OF THE INVENTION

This application claims benefit of priority of U.S. Provisional Application No. 63/013,026, filed Apr. 21, 2020 and U.S. Provisional Application No. 63/159,263, filed Mar. 10, 2021, which are hereby incorporated by reference in their entirety.

### I. FIELD OF THE INVENTION

This invention relates to the field of biotechnology and therapeutic treatment methods.

### II. BACKGROUND

Tremendous advances were made in cancer therapy in the past decade through the use of targeted therapy and immune therapy. By blocking immune inhibitory ligand-receptor interactions involving CTLA-4 and PD-1, checkpoint blockade immunotherapy relieves T lymphocytes of major inhibitory signals, thus potentiating underlying T cell-mediated anti-tumor immune activity. However, ubiquitous relief of inhibitory signals systemically can also activate T lymphocytes reactive against self-antigens, leading to loss of self-tolerance and immune-related adverse events. Patients who develop high-grade toxicities commonly require either temporary or permanent discontinuation of treatment, and may require prolonged periods of heavy immunosuppression in order to manage their toxicities. The high frequency of developing severe to life threatening toxicity to anti-CTLA-4 and/or anti-PD-1 therapy and the unpredictability with respect to whether a patient will respond has become a limiting factor for clinicians to prescribe this form of therapy.

While some factors associated with patient response to immune checkpoint inhibitor therapy have been discovered, there is a need in the art for predictors of toxicity due to immune checkpoint blockade therapy and predictors of responders to immune checkpoint blockade therapy. Stratifying patients into those that are likely and unlikely to respond to checkpoint blockade therapy, based on one or more biomarkers, will provide for more effective and therapeutic treatment methods for patients, since patients can be provided with the most effective therapy before further spreading of the disease.

### SUMMARY OF THE INVENTION

The current disclosure provides for novel therapeutic methods by identifying cancer patient populations that may be treated effectively by immunotherapies. Accordingly, aspects of the disclosure relate to a method of treating cancer in a subject comprising administering to the subject immune checkpoint blockade (ICB) therapy after the subject has been determined to have increased expression of CXCL13 in a biological sample from the subject. Further aspects relate to a method for predicting a response to ICB therapy in a subject having cancer, the method comprising: (a) determining the expression level of CXCL13 in a sample from the subject; (b) comparing the expression level of CXCL13 in a sample from the subject to a control; and (c) predicting that the subject will respond to the ICB therapy after (i) an increased expression level of CXCL13 is detected in a biological sample from the subject as compared to a control, wherein the control represents an expression level of CXCL13 in a biological sample from a subject that has been determined to not respond to ICB therapy; or (ii) a non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to respond to ICB therapy; or (d) predicting that the subject will not respond to the ICB therapy after (i) a decreased or non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to not respond to ICB therapy; or (ii) a decreased expression level of CXCL13 is detected in a biological sample from the subject as compared to a control, wherein the control represents an expression level of CXCL13 in a biological sample from a subject that has been determined to respond to ICB therapy. Yet further aspects relate to a method comprising detecting CXCL13 in a biological sample from a subject with cancer.

Aspects of the disclosure also relate to a method of treating cancer in a subject comprising administering to the subject immune checkpoint blockade (ICB) therapy after the subject has been determined to have increased expression of CXCL13 in a biological sample from the subject; wherein the subject is one that harbors a mutation in at least one allele of the ARID1A gene that results in at least partial loss of protein expression or function. Further aspects relate to a method for predicting a response to ICB therapy in a subject having cancer and having a mutation in at least one allele of the ARID1A gene that results in at least partial loss of protein expression or function, the method comprising: (a) determining the expression level of CXCL13 in a sample from the subject; (b) comparing the expression level of CXCL13 in a sample from the subject to a control; and (c) predicting that the subject will respond to the ICB therapy after (i) an increased expression level of CXCL13 is detected in a biological sample from the subject as compared to a control, wherein the control represents an expression level of CXCL13 in a biological sample from a subject that has been determined to not respond to ICB therapy; or (ii) a non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to respond to ICB therapy; or (d) predicting that the subject will not respond to the ICB therapy after (i) a decreased or non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to not respond to ICB therapy; or (ii) a decreased expression level of CXCL13 is detected in a biological sample from the subject as compared to a control, wherein the control represents an expression level of CXCL13 in a biological sample from a subject that has been determined to respond to ICB therapy. Yet further aspects provide for a method comprising detecting CXCL13 in a biological sample from a subject with cancer; wherein the subject is one that harbors a mutation in at least one allele of the ARID1A gene that results in at least partial loss of protein expression or function.

The cancer may be bladder cancer. The bladder cancer may be further defined as urothelial cancer. In some aspects, the cancer is cholangiocarcinoma and/or a cancer of the biliary tract. The cancer may also be a cancer described herein. The subject may be one that has been diagnosed with cancer. The bladder cancer may be further defined as bladder cancer with high-density tertiary lymphoid structure. The subject may be further defined as one that is ARID1A mutant. The ARID1A mutant subject is one that has harbors a mutation in at least one allele of the ARID1A gene that results in at least partial loss of protein expression or function. In some aspects, the expression level of CXCL13 and the ARID1A mutation status was determined in the subject and wherein the ARID 1A mutation status was determined prior to the CXCL13 expression level. In some aspects, the expression level of CXCL13 was determined prior to the expression level or mutation status of ARID1A. The expression level and/or mutation status of CXCL13 and/or ARID1A may be determined by NanoString analysis. In some aspects, the subject was determined to a mutation in at least one allele of the ARID1A gene by NanoString analysis. The expression level or mutation status of ARID1A may be determined prior to the expression level of CXCL13. Accordingly, the methods of the disclosure may first comprise stratifying the patient population based on ARID1A mutation status and then further stratifying the ARID1A mutant patient population based on CXCL13 expression levels. The expression level of CXCL13 may be detected, evaluated, or determined by performing immunohistochemistry to detect CXCL13 expression in the biological sample from the subject. The subject may be a subject that has loss of expression of the ARID1A protein. The subject may also be a subject that has loss of function in the ARID1A protein. The mutation of the ARID1A gene may be in one copy of the subject genome or in two copies of the ARID 1A gene in the subject's genome. The mutation may be further characterized as a loss of function, missense, deletion, or insertion. The subject may be one that is determined to have decreased expression of ARID1A protein. In some aspects, the expression of ARID1A is statistically significantly lower than a control level of ARID1A expression. The expression of ARID1A may be at least 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000% higher (or any derivable range therein) than a control level of ARID1A expression. The control level of ARID1A expression may be a level of expression from a subject that is wild-type for the ARID1A gene or one that has been determined to not respond to ICB therapy.

The term “NanoString analysis” refers to NanoString-based nucleic acid detection and quantification. NanoString analysis uses molecular “barcodes” and microscopic imaging to detect and count up to several hundred unique transcripts in one hybridization reaction. For example, a NanoString process can utilize two different probes, both recognizing different portions of the same DNA or RNA target The first probe, called the capture-probe, can be labeled with biotin which immobilizes the molecules of interest onto a counting stand. The second probe called the reporter probe, can carry a unique fluorescent tag composed of 6 fluorochromes of four possible colors linked together. Each probe's particular color combination can be specific for its target molecule. This unique color code can give the technique a very high sensitivity and allows analysis of quantity-limited biological samples. These two nucleic acid specific probes can interact in solution with the lysates derived from the biologic sample. Each of the probes can bind to the targeted nucleic acids in solution and then are immobilized onto reading slides. These slides can then be washed to remove unbound fluorescent probes and can then be aligned and read by the nCounter instrument. This is further described in Geiss et al., Nature Biotechnology. 26 (3): 317-25, which is herein incorporated by reference.

In some aspects, the expression of the CXCL13 gene or protein is high as compared to a control. The expression of CXCL13 may be at least or is at most 1, 2, 3, 4, 5, 6, 7, or 8 deviations (or any derivable range therein) from the control. In some aspects, the expression of CXCL13 is statistically significantly higher than a control level of CXCL13 expression. The expression of CXCL13 may be at least 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000% higher (or any derivable range therein) than a control level of CXCL13 expression. The control may comprise a cut-off value or a normalized value. The increased expression level of CXCL13 may comprise a level of expression or normalized level of expression that is determined to be increased as compared to a control. The control may represent an expression level of CXCL13 in a biological sample from a subject that has been determined to not respond to ICB therapy. The subject may be one that was determined to have a level of CXCL13 expression that was not significantly different than a control or within one standard deviation from a control, wherein the control represents an expression level of CXCL13 in a biological sample from a subject that has been determined to respond to ICB therapy. Aspects of the disclosure also relate to comparing the level of expression of CXCL13 to a level of expression in a control. The control may comprise a biological sample from a subject that does not respond to ICB therapy or represents a level of CXCL13 expression in a subject or subjects that have been determined to not respond to ICB therapy. The control may comprise a biological sample from a subject that responds to ICB therapy or represents a level of CXCL13 expression in a subject or subjects that have been determined to respond to ICB therapy.

The cancer may be further defined as recurrent cancer. The subject may be one that has been previously treated for the cancer with an anticancer agent. The previous treatment may comprise a chemotherapeutic. The chemotherapeutic may comprise at least one platinum-based chemotherapeutic agent. The platinum-based chemotherapeutic agent may comprise one or more of cisplatin, carboplatin, and oxaliplatin. The subject may be one that has been determined to be non-responsive to the previous therapy. The subject may be one that has been determined to be a candidate for ICB therapy. The subject may be one that is currently being treated with ICB therapy, has received at least one ICB therapy, or wherein the subject has not been treated with ICB therapy.

The biological sample may comprise a tumor sample or a biopsy sample. The biological sample may comprise a biopsy comprising tumor cells and/or components of the tumor micro environment. In some aspects, the biological sample comprises a biological sample described herein.

The ICB therapy may comprise a monotherapy or a combination ICB therapy. The term ICB monotherapy relates to a therapy that includes one ICB therapeutic agent, excludes all other ICB therapeutic agents, but does not exclude other non-ICB therapeutic agents. The ICB therapy may comprise an inhibitor of PD-1, PDL1, PDL2, CTLA-4, B7-1, and/or B7-2. The ICB therapy may comprise an anti-PD-1 monoclonal antibody and/or an anti-CTLA-4 monoclonal antibody. The ICB therapy may comprise one or more of nivolumab, pembrolizumab, pidilizumab, ipilimumab, and tremelimumab. In some aspects, the ICB therapy comprises or consists of nivolumab monotherapy. In some aspects, the ICB therapy comprises or consists of nivolumab and ipilimumab combination therapy.

The method may further comprise administering at least one additional anticancer treatment. The at least one additional anticancer treatment may include surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

The method may comprise or further comprise treating the subject predicted to respond to ICB therapy with ICB therapy. The method may further comprise determining the expression level or mutation status of ARID1A in the subject. The subject may be one that is predicted to respond to ICB therapy when the expression level of ARID1A is reduced compared to a control or when ARID1A is determined to be mutant. The subject may be one that is predicted to not respond when the expression level of ARID1A is not substantially different than a control level of expression and/or when ARID1A is determined to be wild-type. The mutant ARID1A may comprise a mutation in at least one allele of the ARID1A gene that results in at least partial loss of protein expression or function. In some aspects, the expression level of CXCL13 and/or ARID1A was measured or detected by detecting or measuring mRNA expression or protein expression of CXCL13 and/or ARID1A.

The subject may be a human subject. The subject may also be defined as a mammalian subject. In some aspects, the subject is a mouse, rat, rabbit, dog, cat, horse, or pig. In some aspects, CXCL13 expression is detected in an immunoassay. The immunoassay may be further defined as immunohistochemistry. In some aspects, the CXCL13 expression level is detected, is evaluated, or was determined in a subject by detecting CXCL13 expression by immunohistochemical analysis of tissue samples from the subject.

The subject may be one that has been determined to have decreased expression of ARID1A. The subject may be one that has been determined to have increased expression of CXCL13 and/or decreased expression of ARID1A by detecting or measuring mRNA expression or protein expression of CXCL13 and/or ARID1A. The method may further comprise measuring the expression level of CXCL13 and/or ARID1A in a biological sample from the subject. Measuring the expression level may comprise detecting or measuring mRNA expression. The methods of the disclosure may involve, but not be limited to, next generation sequencing, single-molecule real-time sequencing, mass spectrometry, digital color-coded barcode technology analysis, microarray expression profiling, quantitative PCR, reverse transcriptase PCR, reverse transcriptase real-time PCR, quantitative real-time PCR, end-point PCR, multiplex end-point PCR, cold PCR, ice-cold PCR, in situ hybridization, Northern hybridization, hybridization protection assay (HP A), branched DNA (bDNA) assay, rolling circle amplification (RCA), single molecule hybridization detection, invader assay, and/or Bridge Litigation Assay. Other non-limiting amplification methods may include real-time PCR (quantitative PCR (q-PCR)), digital PCR, nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In some aspects, the expression level of the mRNA of ARID1A and/or CXCL13 is measured or was determined. In some aspects, the expression level of the ARID1A and/or CXCL13 protein is measured or was determined.

The subject may be one that is determined to have a higher expression level than the control. The subject may be one that is determined to have a lower expression level than the control. In the methods of the disclosure, the expression level may be determined to be at least or determined to be at most 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000% higher or lower (or any derivable range therein) than a control level of expression. In some aspects, the subject is determined to have a level of expression that is not significantly different than the control.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-F: Genomic mutation of ARID1A correlates with improved OS in patients with mUCC receiving immune checkpoint therapy. (A) An “oncoplot” showing the 34 commonly mutated genes in bladder cancer. Each column represents a patient tumor sample and each row represents a different gene. The numbers on the left side represent the percentage of mUCC samples carrying mutation of each specific gene. The top barplot indicates the frequency of mutations for each patient. ARID1A is the only gene that has significantly higher mutation frequency in the CR/PR/SD group (N=11) than in the PD group (N=13) (*, Fisher's exact test p-value=0.031). (B) Representative plot of ARID1A gene expression in patients with no ARID1A mutation (ARID1A-WT) (N=20) and with ARID1A mutation (ARID1A mutant) (N=4), **p<0.01. (C) Stack bar plot showing the frequencies of patients with no ARID1A mutation (WT) and with ARID1A mutation (mutant) in the CR/PR/SD group (N=57) and in the PD group (N=61) in the CheckMate 275 trial. P value from logistic regression with response groups (CR/PR/SD vs PD) as dependent variable and ARID1A mutation status as the independent variable. (D) Stack bar plot showing the frequencies of patients with no ARID1A mutation (WT) and with ARID1A mutation (mutant) in the CR/PR/SD group (N=104) and in the PD group (N=133) in the IMvigor 210 trial. P value from logistic regression with response groups (CR/PR/SD vs PD) as dependent variable and ARID1A mutation status as the independent variable. (E) Kaplan-Meier plot showing overall survival (OS) (p=0.03) in patients with mUCC divided into ARID1A-WT (those with no ARID1A mutation) and ARID1A-mutant (those with ARID1A mutation) (N=139, AR/D/A-WT=100, AR/D/A-mutant=39) in the CheckMate 275 trial. (F) Kaplan-Meier plot showing overall survival (OS) (p=0.03) in patients with mUCC divided into ARID1A-WT (those with no ARID1A mutation) and AR/DIA-mutant (those with ARID1A mutation) (N=275, AR/D/A-WT=213, ARID1A-mutant=62) in the IMvigor 210 trial.

FIG. 2A-F. Expression of CXCL13 in the baseline tumor tissues correlated with improved overall survival. (A) Quantification of expression of indicated markers via Immunohistochemistry (IHC) analysis on patient tumor samples (N=31) with Responders (CR/CP/SD)=11 and Non-responders (PD)=20. IM=margin, CT=center. (B) Representative heat map and fold difference of the 12 TLS signature genes using GEP of baseline mUCC tumor specimens (N=21) using customized 739—gene NanoString panel. (C) Representative box plot of CXCL13 gene expression in patients in the CR/PR/SD group and in the PD group in the CheckMate 275 trial, **p<0.01. (D) Representative box plot of CXCL13 gene expression in patients in the CR/PR/SD group (N=104) and in the PD group (N=133) in the IMvigor 210 trial, ****p<0.0001. (E) Kaplan-Meier estimates of overall survival (OS) in patients in the CheckMate 275 trial, stratified according to the 33rd and 66th percentile of CXCL13 expression values. (F) Kaplan-Meier estimates of overall survival (OS) in patients in the IMvigor 210 trial, stratified according to the 33rd and 66th percentile of CXCL13 expression values.

FIG. 3A-C. (A) Kaplan-Meier estimates of overall survival (OS) in patients in the IMvigor 210 trial, stratified according to ARID1A mutation status and 50th percentile of CXCL13 expression values. (B) Predicted log hazard curves demonstrating associations between CXCL13 expression at various ARID1A mutation status and PFS or OS. Hazard curve estimates are from the Cox PH model with linear predictors CXCL13 and ARID1A mutation status and CXCL13:ARID1A mutation status interaction. Shaded areas give 95% pointwise confidence intervals for the hazard curves. Hazard curve estimates were scaled to be zero at the median of CXCL13 score. (C) Graphical summary demonstrating ARID1A mutation plus CXCL13 expression acting as combinatorial biomarkers to predict responses to immune checkpoint therapy in Urothelial Carcinoma.

FIG. 4A-B. CONSORT diagram describing the number of samples used for specific studies in the Checkmate 275 cohort (A) and IMvigor210 (B).

FIG. 5A-C: (A) Graph representing tumor weight (mg) of ARID1A knockdown and scrambled control MB49 cells with and without anti-PD-1 treatment (n=5 in each group,*p<0.05, ** p<0.01, ***p<0.001 determined by Student's t test). (B) Representative heat map indicating transcriptomic profiles of ARID1A knockdown and scrambled control MB49 cells. (C) Gene Set Enrichment (GSEA) plot of the upregulated and downregulated pathways in ARID1A knockdown and scrambled control MB49 cells.

FIG. 6A-G. (A) Representative plot of relative TMB level in patients with no ARID1A mutation (ARID1A-WT) (N=100) and with ARID1A mutation (ARID1A mutant) (N=39) in the CheckMate 275 trial, TMB was log transformed. (B) Representative plot of relative TMB level in patients with no ARID1A mutation (ARID1A-WT) (N=213) and with ARID1A mutation (ARID1A mutant) (N=62) in the IMvigor 210 trial, TMB was log transformed and scaled. (C) Representative plot of relative TMB level in patients with no ARID1A mutation (ARID1A-WT) (N=303) and with ARID1A mutation (ARID1A mutant) (N=101) in the TCGA BLCA cohort, TMB was log transformed and scaled. (D) Representative plot of relative TGFB1 level in patients with no ARID1A mutation (ARID1A-WT) (N=213) and with ARID1A mutation (ARID1A mutant) (N=62) in the IMvigor 210 trial, TGFB1 expression reads was log transformed and scaled. (E) Representative plot of TGFB1 expression in patients with no ARID1A mutation (ARID1A-WT) (N=303) and with ARID1A mutation (ARID1A mutant) (N=101) in the TCGA BLCA cohort, TGFB1 expression reads was log transformed and scaled. (F) Percentage of PD-L1 positive tumor cells and immune cells in patients with no ARID1A mutation (ARID1A-WT) and with ARID1A mutation (ARID1A mutant) in the CheckMate 275 biomarker cohort. (G) Stack bar plot showing the frequencies of patients with different PDL1 levels in the immune cells in the no ARID1A mutation (WT) group and with ARID1A mutation (mutant) group in the IMvigor 210 trial. P value from Pearson's Chi-squared test.

FIG. 7. Forest plot of the Harzard Ratio and its 95% confidence interval from the univariable Cox Proportional-Hazards Model to test the association of top 20 mutated genes (Mutant vs WT) and overall survival in the IMvigor 210 trial.

FIG. 8A-D. (A) Composite image (panel a) showing positive expression for CD20 cells (white, panel b), CD8 (red, panel c) and CD4 (yellow, panel d). (B-C) Quantification of Ratio of TLS/total area and TLS density in both non-responders (blue) and responders (red). Each dot represents individual sample. ****p<0.0001. (D) Representative heat map of transcriptome profiling using GEP of baseline mUCC tumor specimens (N=21; R=8, NR=13) using customized 739—gene Nanostring panel.

FIG. 9A-D. (A) Graph representing the tumor weight (mg) of WT and CXCL13^{-/-} bearing MB49 tumors with and without anti-PD-1 treatment. (B) Heat map depicting the specific cell clusters and defined markers in each cluster identified through CyTOF and analyzed using FlowSOM clustering algorithm. Arrow indicate the expression of markers in Cluster 2. (C) Graph depicting frequency of intra-tumoral CD3⁺CD8⁺ICOS⁺GzmB⁺PD-1⁺ T cells (cluster 13). (n=5 in each group,*p<0.05, **p<0.01, ***p<0.001 determined by Student's t test). Data are representative of three independent experiments. (D) Scatter plot of the association of CXCL13 level with the expression levels of CD8A, CD4, CD19, and IFNG in the IMvigor 210 trial (n=348). The blue line is the regression line. The R2 and p value are from linear regression.

### DETAILED DESCRIPTION OF THE INVENTION

Immune checkpoint therapy (ICT) can produce durable anti-tumor responses in metastatic urothelial carcinoma (mUCC); however, the responses are not universal. Despite multiple approvals of ICT in mUCC, there remains a lack of predictive biomarkers to guide patient selection. The identification of biomarkers may require interrogation of both the tumor mutational status and the immune microenvironment. Through multi-platform immuno-genomic analyses of baseline tumor tissues, the inventors identified the genomic mutation of ARID1A in tumor cells and expression of immune cytokine CXCL13 in the baseline tumor tissues as two predictors of clinical responses in a discovery cohort (N=31). Further, reverse translational studies revealed that CXCL13^{-/-} tumor-bearing mice were resistant to ICT whereas ARID1A knockdown enhanced sensitivity to ICT in a murine model of bladder cancer. Next, the inventors tested the clinical relevance of genomic mutation of ARID1A and baseline CXCL13 expression in two independent confirmatory cohorts (CheckMate275 and IMvigor210). It was found that genomic mutation of ARID1A and expression of CXCL13 in the baseline tumor tissues correlated with improved overall survival (OS) in both the confirmatory cohorts (CheckMate275, CXCL13 data, N=217; ARID1A data, N=139, and IMvigor210, CXCL13 data, N=348; ARID1A data, N=275). Importantly, recognizing the importance of both biomarkers, the inventors interrogated CXCL13 expression and ARID1A mutation for synergistic performance in predicting response to ICT in CheckMate275 and IMvigor210. Combination of the 2 biomarkers in baseline tumor tissues correlated with improved OS compared to a single biomarker. Cumulatively, the inventors studies revealed that CXCL13 and/or ARID1A may improve predictive outcomes for patients receiving ICT and enable patient selection for ICT.

### I. Bladder Cancer

Aspects of the disclosure relate to methods of treating, detecting, or prognosing bladder cancer or a response to a bladder cancer therapy. The bladder cancer may refer to any type of bladder cancer. In certain aspects, the bladder cancer comprises urothelial cancer. In some aspects, the bladder cancer comprises urothelial carcinoma. Urothelial carcinoma may also be referred to as transitional cell carcinoma or TCC. In some aspects, the bladder cancer comprises squamous cell carcinoma. In some aspects, the bladder cancer comprises adenocarcinoma. In some aspects, the bladder cancer comprises sarcoma of the bladder. In some aspects, the bladder cancer comprises small cell bladder cancer.

The bladder cancer may be further defined as one of noninvasive, non-muscle-invasive, or muscle-invasive. The bladder cancer may be of a certain stage or stage group, as shown in the table below:

| | Stage | |
|---|---|---|
| Stage | Group | Description |
| 0a | Ta; | The cancer is a non-invasive papillary carcinoma |
| | N0; M0 | (Ta). It has grown toward the hollow center of the |
| | | bladder but has not grown into the connective |
| | | tissue or muscle of the bladder wall. It has not |
| | | spread to nearby lymph nodes (N0) or distant |
| | | sites (M0). |
| 0is | Tis; | The cancer is a flat, non-invasive carcinoma |
| | N0; M0 | (Tis), also known as flat carcinoma in situ (CIS). |
| | | The cancer is growing in the inner lining layer |
| | | of the bladder only. It has not grown inward |
| | | toward the hollow part of the bladder, nor has |
| | | it invaded the connective tissue or muscle of |
| | | the bladder wall. It has not spread to nearby |
| | | lymph nodes (N0) or distant sites (M0). |
| I | TI; N0; | The cancer has grown into the layer of connective |
| | M0 | tissue under the lining layer of the bladder, |
| | | but has not reached the layer of muscle in the |
| | | bladder wall (T1). The cancer has not spread to |
| | | nearby lymph nodes (N0) or to distant sites (M0). |
| II | T2a or | The cancer has grown into the inner (T2a) or outer |
| | T2b; | (T2b) muscle layer of the bladder wall, but it has |
| | N0; | not passed completely through the muscle to reach |
| | M0 | the layer of fatty tissue that surrounds the |
| | | bladder. The cancer has not spread to nearby |
| | | lymph nodes (N0) or to distant sites (M0). |
| IIIA | T3a, | The cancer has grown through the muscle layer |
| | T3b or | of the bladder and into the layer of fatty tissue |
| | T4a; | that surrounds the bladder (T3a or T3b). It might |
| | N0; M0 | have spread into the prostate, seminal vesicles, |
| | | uterus, or vagina, but it's not growing into the |
| | | pelvic or abdominal wall (T4a). The cancer has |
| | | not spread to nearby lymph nodes (N0) or to |
| | | distant sites (M0). |
| | OR | |
| | T1-4a; | The cancer has: |
| | N1; M0 | grown into the layer of connective tissue under |
| | | the lining of the bladder wall (T1), OR |
| | | into the muscle layer of the bladder wall (T2), OR |
| | | into the layer of fatty tissue that surrounds the |
| | | bladder, (T3a or T3b) OR |
| | | it might have spread into the prostate, seminal |
| | | vesicles, uterus, or vagina, but it's not growing |
| | | into the pelvic or abdominal wall (T4a). |
| | | AND the cancer has spread to 1 nearby lymph node |
| | | in the true pelvis (N1). It has not spread to |
| | | distant sites (M0). |
| IIIB | T1-T4a; | The cancer has: |
| | N2 or | grown into the layer of connective tissue under |
| | N3; M0 | the lining of the bladder wall (T1), OR |
| | | into the muscle layer of the bladder wall (T2), OR |
| | | into the layer of fatty tissue that surrounds the |
| | | bladder (T3a or T3b), OR |
| | | it might have spread into the prostate, seminal |
| | | vesicles, uterus, or vagina, but it's not growing |
| | | into the pelvic or abdominal wall (T4a). |
| | | AND the cancer has spread to 2 or more lymph nodes |
| | | in the true pelvis (N2) or to lymph nodes along |
| | | the common iliac arteries (N3). It has not spread |
| | | to distant sites (M0). |
| IVA | T4b; | The cancer has grown through the bladder wall into |
| | Any N; | the pelvic or abdominal wall (T4b). It might or |
| | M0 | might not have spread to nearby lymph nodes (Any N). |
| | | It has not spread to a distant sites (M0). |
| | OR | |
| | Any T; | The cancer might or might not have grown through |
| | Any N; | the wall of the bladder into nearby organs (Any T). |
| | M1a | It might or might not have spread to nearby lymph |
| | | nodes (Any N). It has spread to distant lymph |
| | | nodes (M1a). |
| IVB | Any T; | The cancer might or might not have grown through |
| | Any N; | the wall of the bladder into nearby organs (Any T). |
| | M1b | It might or might not have spread to nearby lymph |
| | | nodes (Any N). It has spread to 1 or more distant |
| | | organs, such as the bones, liver, or lungs (M1b). |

The bladder cancer may comprise stage 0a, 0is, I, II, IIIA, IIIB, WA, or IVB bladder cancer. In some aspects, the bladder cancer excludes stage 0a, 0is, I, II, IIIA, IIIB, IVA, or IVB bladder cancer.

### II. Immunotherapy

The methods of the disclosure may comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

A. Immune Checkpoint Blockade Therapy

Aspects of the disclosure may include administration of immune checkpoint blockade therapy, which are further described below.

1. PD-1, PDL1, and PDL2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

The PD-1 inhibitor may be a molecule that inhibits the binding of PD-1 to its ligand binding partners. In a specific aspect, the PD-1 ligand binding partners are PDL1 and/or PDL2. A PDL1 inhibitor may be a molecule that inhibits the binding of PDL1 to its binding partners. In a specific aspect, PDL1 binding partners are PD-1 and/or B7-1. The PDL2 inhibitor may be a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

The PD-1 inhibitor may be an anti-PD-1 antibody (e.g., a human antibody, a humanized antibody, or a chimeric antibody). The anti-PD-1 antibody may be selected from the group consisting of nivolumab, pembrolizumab, and pidilizumab. The PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence)). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

The ICB therapy may comprise a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the ICB therapy comprises a PDL2 inhibitor such as rHIgM12B7.

The inhibitor may comprise the heavy and light chain CDRs or VRs of nivolumab, pembrolizumab, or pidilizumab. Accordingly, the inhibitor may comprise the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. The antibody may be one that competes for binding with and/or binds to the same epitope on PD-1, PDL1, or PDL2 as the above-mentioned antibodies. The antibody may have at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the ICB therapy comprises an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as an ICB therapy in the methods and compositions of the disclosure is ipilimumab (also known as 10D1, MDX-010, MDX-101, and Yervoy®) or antigen binding fragments and variants thereof (see, e.g., WOO 1/14424).

The inhibitor may comprise the heavy and light chain CDRs or VRs of tremelimumab or ipilimumab. Accordingly, the inhibitor may comprise the CDR1, CDR2, and CDR3 domains of the VH region of nivolumab, pembrolizumab, pidilizumab, ipilimumab or tremelimumab, and the CDR1, CDR2 and CDR3 domains of the VL region of nivolumab, pembrolizumab, pidilizumab, ipilimumab or tremelimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

B. Inhibition of Co-Stimulatory Molecules

The immunotherapy may comprise an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD4OLG), GITR (TNFRSF18), and combinations thereof. Inhibitors include inhibitory antibodies, polypeptides, compounds, and nucleic acids.

C. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

D. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

E. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

F. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### III. Additional Therapies

The current methods and compositions of the disclosure may include one or more additional therapies or include patients who have previously been treated with one or more additional therapies known in the art and/or described herein. In some aspects, the additional therapy comprises an additional anticancer treatment. Examples of such treatments are described herein, such as the immunotherapies described herein or the additional therapy types described in the following.

A. Oncolytic Virus

In some aspects, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

B. Polysaccharides

In some aspects, the additional therapy comprises polysaccharides. Certain compounds found in mushrooms, primarily polysaccharides, can up-regulate the immune system and may have anticancer properties. For example, beta-glucans such as lentinan have been shown in laboratory studies to stimulate macrophage, NK cells, T cells and immune system cytokines and have been investigated in clinical trials as immunologic adjuvants.

C. Neoantigens

In some aspects, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

D. Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dacarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-a), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluorouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2- to 10,000-fold less than the chemotherapeutic effective dose of the chemotherapeutic agent. For example, the chemotherapeutic agent may be administered in an amount that is about 20-fold less, about 500-fold less or even about 5000-fold less than the effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

In some aspects, the chemotherapy comprises mitomycin. In some aspects, the chemotherapy comprises gemcitabine. In some aspects, the chemotherapy comprises valrubicin. In some aspects, the chemotherapy comprises cisplatin. In some aspects, the chemotherapy comprises cisplatin plus fluorouracil (5-FU). In some aspects, the chemotherapy comprises mitomycin with 5-FU. In some aspects, the chemotherapy comprises gemcitabine and cisplatin. In some aspects, the chemotherapy comprises one or more of dose-dense methotrexate, vinblastine, doxorubicin (Adriamycin), and cisplatin (DDMVAC). In some aspects, the chemotherapy comprises cisplatin, methotrexate, and vinblastine (CMV). In some aspects, the chemotherapy comprises gemcitabine and paclitaxel.

E. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Grays (Gy) and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

F. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anticancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some aspects, the additional anticancer therapy comprises surgery. The type of surgery done depends on the stage (extent) of the cancer. In some embodiments, the surgery comprises transurethral resection of bladder tumor (TURBT). A transurethral resection of bladder tumor (TURBT) or a transurethral resection (TUR) is often used to find out if someone has bladder cancer and, if so, whether the cancer has spread into (invaded) the muscle layer of the bladder wall. In some aspects, the surgery comprises cystectomy. When bladder cancer is invasive, all or part of the bladder may need to be removed. This operation is called a cystectomy. In some aspects, the cystectomy may be further defined as partial or radical. In some aspects, the surgery may be laparoscopic.

G. Intravesical Therapy

With intravesical therapy, the clinician may put a liquid drug directly into the bladder rather than giving it by mouth or injecting it into the blood. Intravesical chemotherapy or immunotherapy may be used in aspects of the disclosure. It may be done on a specific regimen such as, for example, once a week for 6 weeks, and may be repeated for another 6 weeks if needed. After a 4- to 6-week break, maintenance treatments are then done for at least 1 year. The intravesical therapy may be immunotherapy or chemotherapy. In some aspects, the anticancer therapy comprises Bacillus Calmette-Guerin or BCG.

H. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

In some aspects, the additional therapy may be a targeted therapy. Examples of targeted therapies include FGFR inhibitors such as Erdafitinib (Balversa). In some aspects, the additional therapy comprises Enfortumab vedotin-ejfv (Padcev).

IV. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from esophageal tissue by any of the biopsy methods previously mentioned. In other aspects the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some aspects, the sample comprises a fractionated sample, such as a blood sample that has been fractionated by centrifugation or other fractionation technique. The sample may be enriched in white blood cells or red blood cells. In some aspects, the sample may be fractionated or enriched for leukocytes or lymphocytes. In some aspects, the sample comprises a whole blood sample.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one aspect, the sample is a fine needle aspirate of a esophageal or a suspected esophageal tumor or neoplasm. In some cases, the fine needle aspirate sampling procedure may be guided by the use of an ultrasound, X-ray, or other imaging device.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

V. Cancer Monitoring

In certain aspects, the methods of the disclosure may be combined with one or more other cancer diagnosis or screening tests at increased frequency if the patient is determined to be at high risk for recurrence or have a poor prognosis based on the biomarker expression described above.

In some aspects, the methods of the disclosure further include one or more monitoring tests. The monitoring protocol may include any methods known in the art. In particular, the monitoring include obtaining a sample and testing the sample for diagnosis. For example, the monitoring may include endoscopy, biopsy, endoscopic ultrasound, X-ray, barium swallow, a Ct scan, a MRI, a PET scan, laparoscopy, or cancer biomarker testing. In some aspects, the monitoring test comprises radiographic imaging. Examples of radiographic imaging this is useful in the methods of the disclosure includes hepatic ultrasound, computed tomographic (CT) abdominal scan, liver magnetic resonance imaging (MRI), body CT scan, and body MRI.

Methods of the disclosure may further include one or more of a urinalysis, urine cytology, urine culture, or urine tumor marker tests. Different urine tests look for specific substances made by cancer cells. One or more of these tests may be used in the methods of the disclosure. These include the tests called NMP22® (or BladderChek®), BTA Stat®, Immunocyt®, and UroVysion®. Methods of the disclosure also include cystoscopy. In this method, a urologist uses a cystoscope, which is a long, thin, flexible tube with a light and a lens or a small video camera on the end. Fluorescence cystoscopy (also known as blue light cystoscopy) may be done along with routine cystoscopy. For this exam, a light-activated drug is put into the bladder during cystoscopy. It's taken up by cancer cells. When the doctor then shines a blue light through the cystoscope, any cells containing the drug will glow (fluoresce). This can help the doctor see abnormal areas that might have been missed by the white light normally used.

Methods of the disclosure also include the use of transurethral resection of bladder tumor (TURBT). The procedure used to biopsy an abnormal area is a transurethral resection of bladder tumor (TURBT), also known as just a transurethral resection (TUR). During this procedure, the doctor removes the tumor and some of the bladder muscle around the tumor. The removed samples are then sent to a lab to look for cancer. Bladder cancer can sometimes start in more than one area of the bladder (or in other parts of the urinary tract). Because of this, the doctor may take samples from many different parts of the bladder, especially if cancer is strongly suspected but no tumor can be seen. Salt water washings of the inside the bladder may also be collected and tested for cancer cells.

In some aspects, imaging tests are performed or the subject is one that has undergone an imaging test. Imaging tests may use x-rays, magnetic fields, sound waves, or radioactive substances. In some aspects, the imaging test comprises an Intravenous pyelogram (IVP). An intravenous pyelogram (IVP), also called an intravenous urogram (IVU), is an x-ray of all of the urinary system taken after injecting a special dye into a vein. This dye is removed from the bloodstream by the kidneys and then passes into the ureters and bladder. X-rays are done while this is happening. The dye outlines these organs on the x-rays and helps show urinary tract tumors. In some aspects, the imaging test comprises a retrograde pyelogram. For this test, a catheter (thin tube) is put in through the urethra and up into the bladder or into a ureter. Then a dye is injected through the catheter to make the lining of the bladder, ureters, and kidneys easier to see on x-rays. In some aspects, the imaging test comprises computed tomography (CT) scan. A CT scan uses x-rays to make detailed cross-sectional pictures of the body. CT-guided needle biopsy: CT scans can also be used to guide a biopsy needle into a suspected tumor. This can be used to take samples from areas where the cancer may have spread. In some aspects, the imaging test comprises magnetic resonance imaging (MRI) scan. Like CT scans, MRI scans show detailed images of soft tissues in the body. But MRI scans use radio waves and strong magnets instead of x-rays. In some aspects, the imaging test comprises an ultrasound. Ultrasound uses sound waves to create pictures of internal organs. Ultrasound can also be used to guide a biopsy needle into a suspected area of cancer in the abdomen or pelvis. In some aspects, the imaging test comprises a chest x-ray or bone scan. A chest x-ray or bone scan may be done to see if the bladder cancer has spread to the lungs or bone, respectively.

VI. ROC Analysis

In statistics, a receiver operating characteristic (ROC), or ROC curve, is a graphical plot that illustrates the performance of a binary classifier system as its discrimination threshold is varied. The curve is created by plotting the true positive rate against the false positive rate at various threshold settings. (The true-positive rate is also known as sensitivity in biomedical informatics, or recall in machine learning. The false-positive rate is also known as the fall-out and can be calculated as 1-specificity). The ROC curve is thus the sensitivity as a function of fall-out. In general, if the probability distributions for both detection and false alarm are known, the ROC curve can be generated by plotting the cumulative distribution function (area under the probability distribution from —infinity to +infinity) of the detection probability in the y-axis versus the cumulative distribution function of the false-alarm probability in x-axis.

ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

The ROC curve was first developed by electrical engineers and radar engineers during World War II for detecting enemy objects in battlefields and was soon introduced to psychology to account for perceptual detection of stimuli. ROC analysis since then has been used in medicine, radiology, biometrics, and other areas for many decades and is increasingly used in machine learning and data mining research.

The ROC is also known as a relative operating characteristic curve, because it is a comparison of two operating characteristics (TPR and FPR) as the criterion changes. ROC analysis curves are known in the art and described in Metz CE (1978) Basic principles of ROC analysis. Seminars in Nuclear Medicine 8:283-298; Youden WJ (1950) An index for rating diagnostic tests. Cancer 3:32-35; Zweig MH, Campbell G (1993) Receiver-operating characteristic (ROC) plots: a fundamental evaluation tool in clinical medicine. Clinical Chemistry 39:561-577; and Greiner M, Pfeiffer D, Smith RD (2000) Principles and practical application of the receiver-operating characteristic analysis for diagnostic tests. Preventive Veterinary Medicine 45:23-41, which are herein incorporated by reference in their entirety. A ROC analysis may be used to create cut-off values for prognosis and/or diagnosis purposes.

VII. Nucleic Acid Assays

Aspects of the methods include assaying nucleic acids to determine expression or activity levels and/or the presence of CXCL13 expressing cells and/or ARID1A mutant cells in a biological sample. Arrays can be used to detect differences between two samples. Specifically contemplated applications include identifying and/or quantifying differences between RNA from a sample that is normal and from a sample that is not normal, between a cancerous condition and a non-cancerous condition. Also, RNA may be compared between a sample believed to be susceptible to a particular disease or condition and one believed to be not susceptible or resistant to that disease or condition. A sample that is not normal is one exhibiting phenotypic trait(s) of a disease or condition or one believed to be not normal with respect to that disease or condition. It may be compared to a cell that is normal with respect to that disease or condition. Phenotypic traits include symptoms of, or susceptibility to, a disease or condition of which a component is or may or may not be genetic or caused by a hyperproliferative or neoplastic cell or cells.

To determine expression levels of a biomarker, an array may be used. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

Further assays useful for determining biomarker expression include, but are not limited to, nucleic amplification, polymerase chain reaction, quantitative PCR, RT-PCR, in situ hybridization, Northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

A further assay useful for quantifying and/or identifying nucleic acids, such as nucleic acids comprising biomarker genes, is RNAseq. RNA-seq (RNA sequencing), also called whole transcriptome shotgun sequencing, uses next-generation sequencing (NGS) to reveal the presence and quantity of RNA in a biological sample at a given moment in time. RNA-Seq is used to analyze the continually changing cellular transcriptome. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

VIII. Protein Assays

A variety of techniques can be employed to measure expression levels of polypeptides and proteins in a biological sample to determine biomarker expression levels. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining protein expression levels of biomarkers.

In one aspect, antibodies, or antibody fragments or derivatives, can be used in methods such as Western blots, ELISA, flow cytometry, or immunofluorescence techniques to detect biomarker expression such as CXCL13. In some aspects, either the antibodies or proteins are immobilized on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present disclosure. The support can then be washed with suitable buffers followed by treatment with the detectably labeled antibody. The solid phase support can then be washed with the buffer a second time to remove unbound antibody. The amount of bound label on the solid support can then be detected by conventional means.

Immunohistochemistry methods are also suitable for detecting the expression levels of biomarkers. In some aspects, antibodies or antisera, including polyclonal antisera, and monoclonal antibodies specific for each marker may be used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Immunological methods for detecting and measuring complex formation as a measure of protein expression using either specific polyclonal or monoclonal antibodies are known in the art. Examples of such techniques include enzyme-linked immunosorbent assays (ELISAs), radioimmunoassays (RIAs), fluorescence-activated cell sorting (FACS) and antibody arrays. Such immunoassays typically involve the measurement of complex formation between the protein and its specific antibody. These assays and their quantitation against purified, labeled standards are well known in the art. A two-site, monoclonal-based immunoassay utilizing antibodies reactive to two non-interfering epitopes or a competitive binding assay may be employed.

Numerous labels are available and commonly known in the art. Radioisotope labels include, for example, 36S, 14C, 1251, 3H, and 1311. The antibody can be labeled with the radioisotope using the techniques known in the art. Fluorescent labels include, for example, labels such as rare earth chelates (europium chelates) or fluorescein and its derivatives, rhodamine and its derivatives, dansyl, Lissamine, phycoerythrin and Texas Red are available. The fluorescent labels can be conjugated to the antibody variant using the techniques known in the art. Fluorescence can be quantified using a fluorimeter. Various enzyme-substrate labels are available and U.S. Pat. Nos. 4,275,149, 4,318,980 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, .beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for Use in Enzyme Immunoassay, in Methods in Enzymology (Ed. J. Langone & H. Van Vunakis), Academic press, New York, 73: 147-166 (1981).

IX. Administration of Therapeutic Compositions

The therapy provided herein may comprise administration of a combination of therapeutic agents, such as a first anticancer therapy and a second anticancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 mg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. Methods of Treatment

Provided herein are methods for treating or delaying progression of cancer in an subject through the administration of therapeutic compositions.

In some aspects, the therapies result in a sustained response in the individual after cessation of the treatment. The methods described herein may find use in treating conditions where enhanced immunogenicity is desired such as increasing tumor immunogenicity for the treatment of cancer.

In some aspects, the individual has cancer that is resistant (has been demonstrated to be resistant) to one or more anticancer therapies. In some aspects, resistance to anticancer therapy includes recurrence of cancer or refractory cancer. Recurrence may refer to the reappearance of cancer, in the original site or a new site, after treatment. In some aspects, resistance to anticancer therapy includes progression of the cancer during treatment with the anticancer therapy. In some aspects, the cancer is at early stage or at late stage.

In some aspects of the methods of the present disclosure, the cancer has low levels of T cell infiltration. In some aspects, the cancer has no detectable T cell infiltrate. In some aspects, the cancer is a non-immunogenic cancer (e.g., non-immunogenic colorectal cancer and/or ovarian cancer). Without being bound by theory, the combination treatment may increase T cell (e.g., CD4+ T cell, CD8+ T cell, memory T cell) priming, activation, proliferation, and/or infiltration relative to prior to the administration of the combination.

The cancer may be a solid tumor, metastatic cancer, or non-metastatic cancer. In certain aspects, the cancer may originate in the bladder.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject diagnosed with cancer).

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

In certain aspects, further cancer or metastasis examination or screening, or further diagnosis such as contrast enhanced computed tomography (CT), positron emission tomography-CT (PET-CT), and magnetic resonance imaging (MRI) may be performed for the detection of cancer or cancer metastasis in patients determined to have a certain gut microbiome composition.

Methods of the disclosure relate to the treatment of subjects with cancer. In some aspects, the methods may be employed with respect to individuals who have tested positive for such cancer, who have one or more symptoms of a cancer, or who are deemed to be at risk for developing such a cancer.

The cancer to be treated may be any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. In some aspects, the cancer to be treated is one of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, haemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), cholangiocarcinoma and cancers of the biliary tract, gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome.

### XI. Kits

Certain aspects of the present invention also concern kits containing compositions of the invention or compositions to implement methods of the invention. In some aspects, kits can be used to evaluate expression levels and/or the presence or absence of cell-surface markers. In certain aspects, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules, detection agents, antibodies or inhibitors, or any value or range and combination derivable therein. In some aspects, there are kits for evaluating expression levels and/or cell surface expression of biomarkers in a cell.

Kits may comprise components, which may be individually packaged or placed in a container, such as a tube, bottle, vial, syringe, or other suitable container means.

Individual components may also be provided in a kit in concentrated amounts; in some aspects, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit aspects. In addition, a kit may include a sample that is a negative or positive control for biomarker expression levels.

It is contemplated that any method or composition described herein can be implemented with respect to any other method or composition described herein and that different aspects may be combined. The claims originally filed are contemplated to cover claims that are multiply dependent on any filed claim or combination of filed claims.

Aspects of the disclosure include kits for analysis of a pathological sample by assessing biomarker expression profile for a sample comprising, in suitable container means, two or more probes or detection agents, wherein the probes or detection agents detect one or more markers identified herein.

### XII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: ARID1A Mutation plus CXCL13 Expression Act as Combinatorial Biomarkers to Predict Responses to Immune Checkpoint Therapy in Urothelial Carcinoma

A. Introduction

Urothelial cancer is the sixth most common cancer in the United States and makes up about 5% of new cancer cases each year (1). The five-year overall survival rate for all stages of urothelial cancer remains between 15 to 20%. Approval of immune checkpoint therapies (ICT) for treatment of metastatic urothelial cancer (mUCC) represents a paradigm shift as it demonstrated durable responses and improved overall survival (2-9). Despite durable anti-tumor responses and multiple approvals, responses are not universal, and there is a lack predictive biomarkers to guide the treatment decisions (10, 11). Therefore, there is a critical need to develop clinically useful biomarkers to determine the optimal patient cohorts.

Although sizeable efforts have been made, the dynamic interaction between the tumor and immune system poses challenges to biomarker development as noted with the development of PD-L1 as a potential biomarker (12, 13). The single biomarker studies either focused on tumor mutations or immune response biomarkers, which may limit predictive power due to lack of integration between areas of tumor cell biology and immune cell responses. To address this need, the inventors interrogated both the immune microenvironment and the tumor mutational status to identify potential combinatorial biomarker to predict response to ICT.

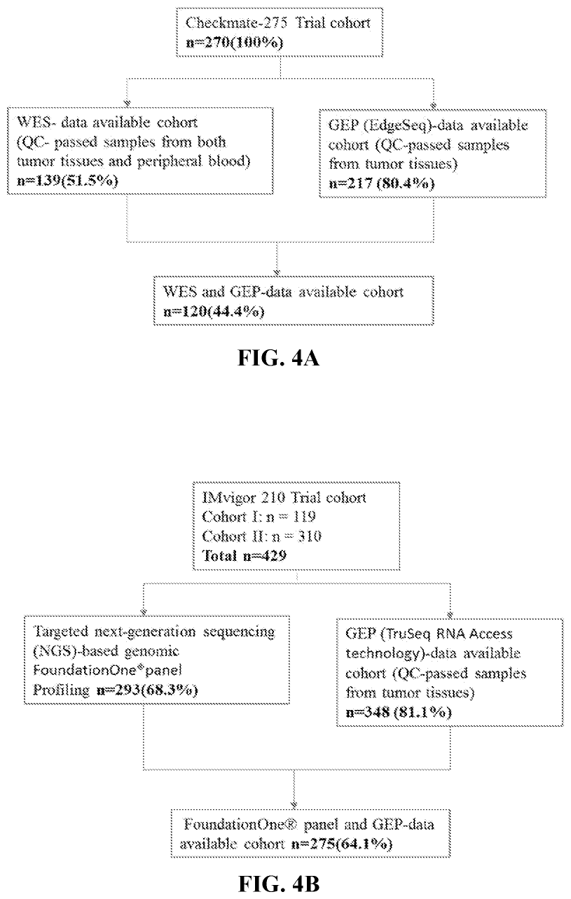

For the discovery cohort, patient samples were obtained at MD Anderson Cancer Center from two clinical trials, a phase 2 study assessing the safety and efficacy of nivolumab in metastatic or surgically unresectable urothelial carcinoma whose disease progressed or recurred despite previous treatment with at least one platinum-based chemotherapy regimen/NCT02387996 and a phase 1/2, open-label study of nivolumab monotherapy or nivolumab combined with ipilimumab in subjects with advanced or metastatic solid tumors/NCT01928394 (14). Response was evaluated using Response Evaluation Criteria In Solid Tumors, version 1.1 (RECIST v1.1). The inventors used CheckMate 275 biomarker cohort (5) and IMvigor 210 (4,15) as the two independent confirmatory cohorts. The consort diagram described the number of samples used to analyze genomic and other correlates in each of the confirmatory cohorts (FIG. 4A-B).

In the discovery cohort, the inventors identified the expression of immune cytokine CXCL13 in the baseline tumor tissues and genomic mutation of ARID1A in tumor cells as two predictors of clinical responses to ICT in patients with mUCC (N=31). Next, the inventors tested the clinical relevance of baseline CXCL13 expression and genomic mutation of ARID1A in two independent confirmatory cohorts (CheckMate275 and IMvigor210). Further, reverse translational studies revealed that CXCL13^{-/-} tumor-bearing mice were resistant to ICT whereas ARID1A knockdown enhanced sensitivity to ICT in a murine model of bladder cancer. The inventors found that the genomic mutation of ARID1A correlated with improved disease control rate and overall survival (OS) in both the confirmatory cohorts (CheckMate275, N=139, and IMvigor210, N=275). Similarly, the inventors noted that expression of CXCL13 in the baseline tumor tissues correlated with improved disease control rate (CR/PR/SD vs PD) and overall survival (OS) in both the confirmatory cohorts (CheckMate275, N=217; and IMvigor210, N=348). Recognizing the importance of both biomarkers, the inventors hypothesized that integration of the genomic and the immunological biomarker would enhance the predictive performance. The inventors interrogated CXCL13 expression and ARID1A mutation for synergistic performance in predicting response to ICT in CheckMate275 and IMvigor210. Combination of both the biomarkers in baseline tumor tissues correlated with improved OS compared to a single biomarker. Cumulatively, this study revealed that the combination biomarkers of CXCL13:ARID1A may improve predictive outcomes for patients receiving ICT and enable patient selection for ICT.

B. Results

### 1. Genomic Mutation of ARID1A Correlates with Improved OS in Patients with mUCC Receiving Immune Checkpoint Therapy

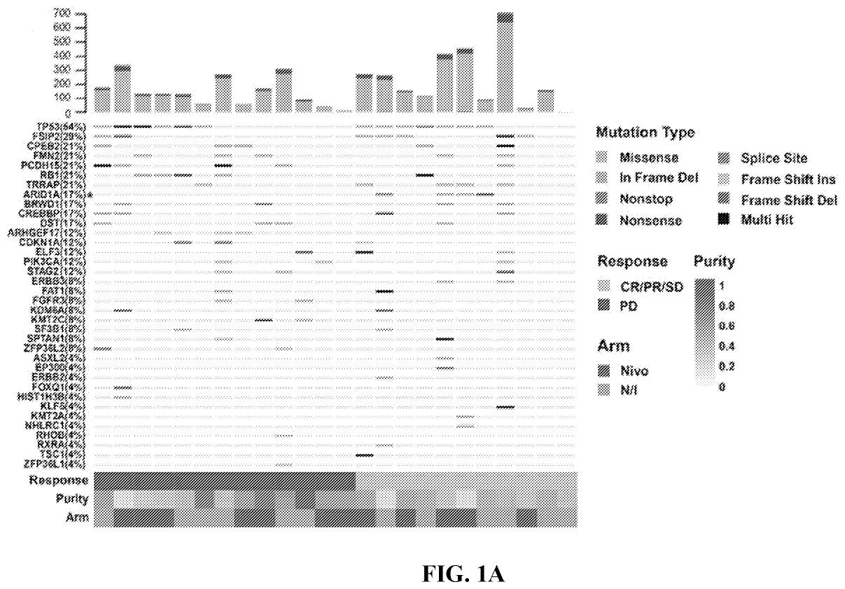

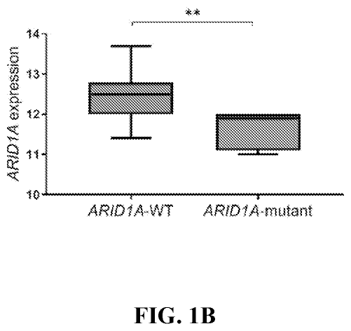

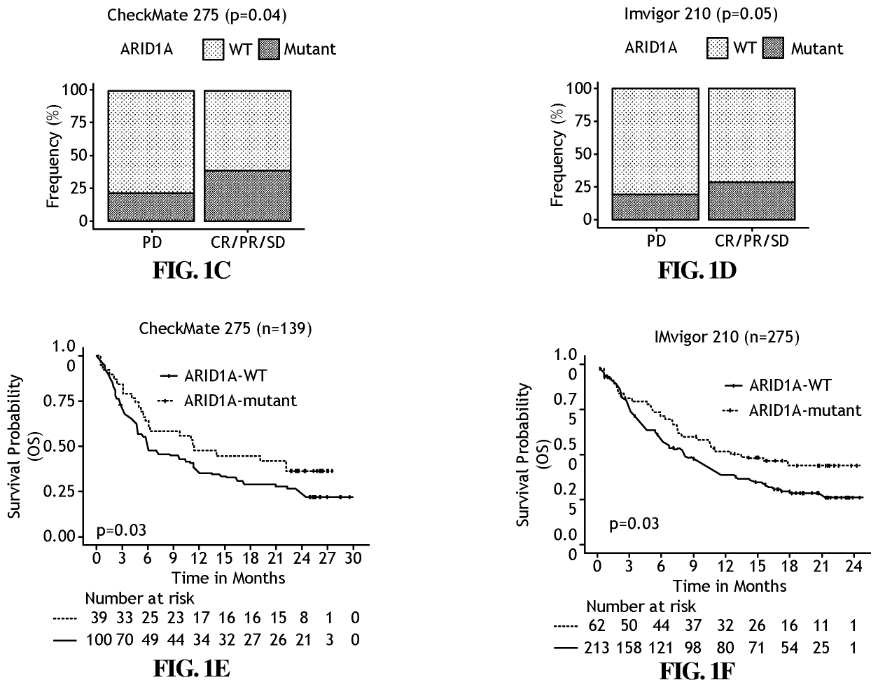

Whole exome sequencing from paired baseline bladder tumor samples and peripheral blood mononuclear cells (as controls) was performed to identify genomic signatures that correlate with clinical benefit to ICT (N=24). The inventors selected the most mutated genes from the TCGA bladder cancer cohort and ordered by decreasing frequency of occurrence. Notably, ARID1A was the only gene in the discovery cohort that was significantly enriched in responders (CP/PR/SD) as compared to non-responders (PD) (p=0.03). Specifically, a total of 4 patient's tumors harbored ARID1A mutations among the 11 responders, whereas no ARID1A mutations were noted in tumors of non-responders (FIG. 1A). The inventors noted that ARID1A mutations result in the downregulation of ARID1A expression (FIG. 1B).

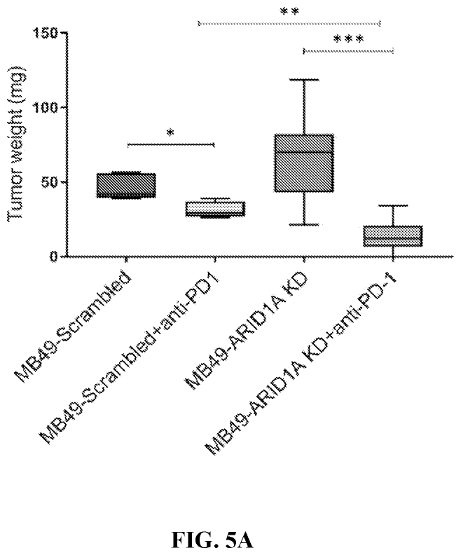

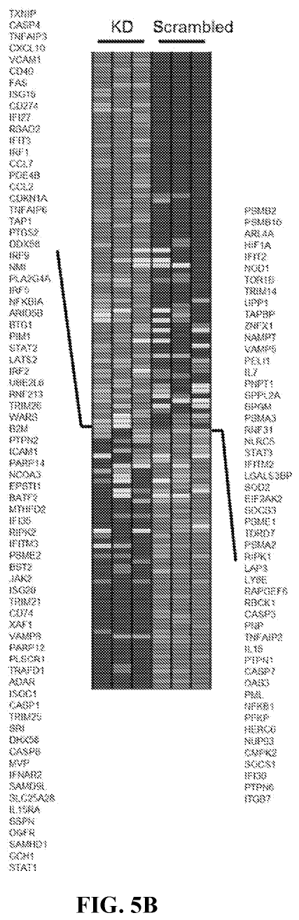

ARID1A is a subunit of SWI/SNF complex required for chromatin remodeling and known to interact with the transcription machinery (16). Previously published preclinical data using cell lines and a murine ovarian tumor model showed that knockdown of ARID1A in ovarian tumor cell lines increased sensitivity to ICT (17). Similarly, the inventors noted that knockdown of ARID1A in MB49 bladder cancer cell lines increased sensitivity to anti-PD1 therapy (FIG. 5A). Further, RNA-sequencing and GSEA analysis of control (scramble) and AR/DIA-knockdown (ARID1A-KD) MB49 cell lines showed that loss of ARID1A led to changes in the expression of distinct pathways regulating immune responses in the tumor. ARID1A KD upregulated interferon response genes and cytokine pathways in the tumor cells (FIG. 5B-C), whereas the DNA repair and the angiogenesis pathways were downregulated (FIG. 5C). Cumulatively, this data suggested that loss of ARID1A could enhance the immunogenicity of the bladder tumor cells.

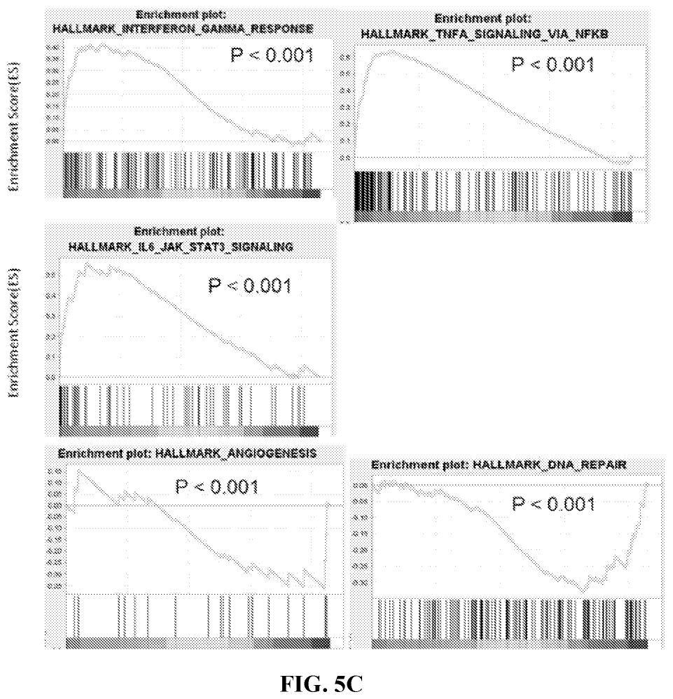

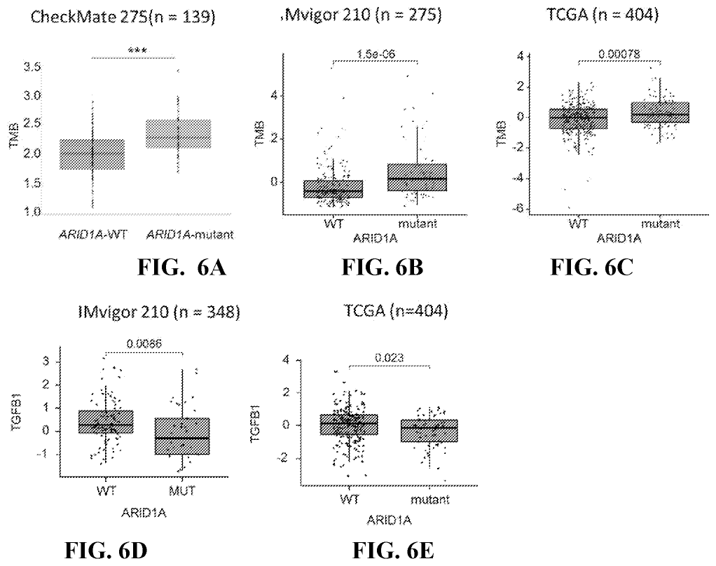

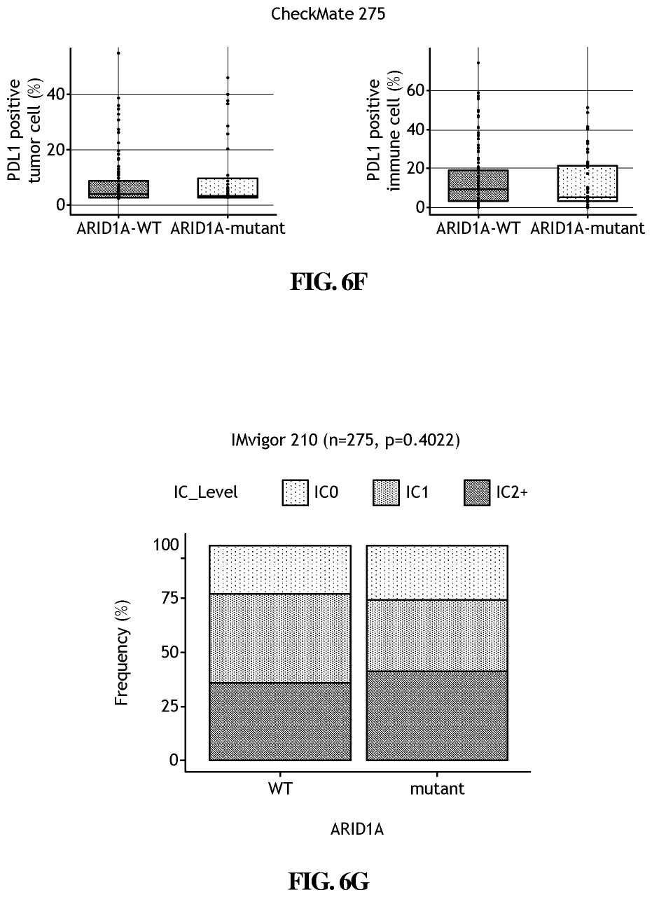

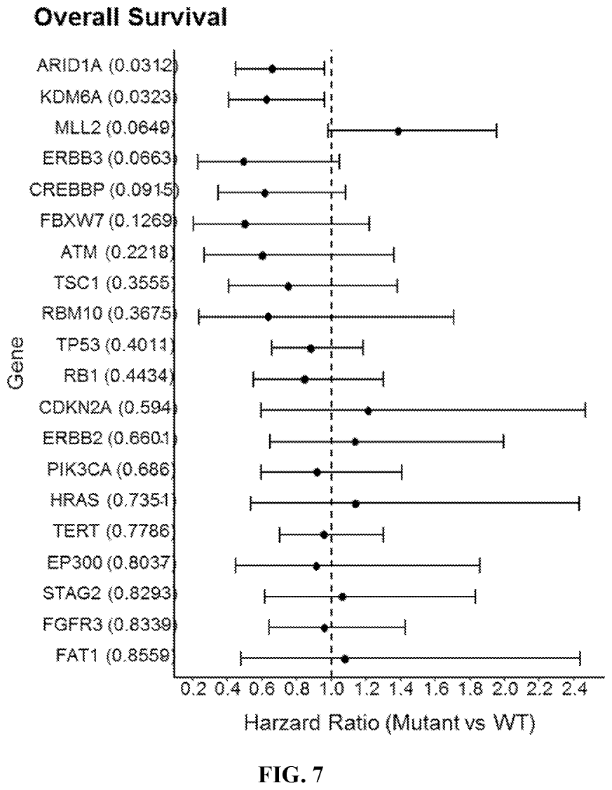

Next, the inventors tested the clinical relevance of ARID1A mutation in patients with mUCC receiving ICT in two independent confirmatory cohorts (CheckMate 275 and IMvigor210). Consistent with previous report (17), the inventors found that patients harboring ARID1A gene mutation have higher tumor mutation burden (TMB) compared to patients without the mutation (FIG. 6A-C). Additionally, the inventors noted that TGFβ1 expression was significantly lower in patients harboring ARID1A gene mutation compared to patients without the mutation (FIG. 6D-E). Both in CheckMate 275 and IMvigor 210, the inventors did not see any difference in PD-L1 expression in tumor or in immune cells based on the presence or absence of ARID1A mutation (FIG. 6F-G). Next, the inventors compared the disease control rate in patients with and without the genomic mutation in ARID1A. The inventors noted that similar to discovery cohort, patients with ARID1A mutations have improved disease control rate compared to the patients without ARID1A mutations in both the confirmatory cohorts (CheckMate 275 and IMvigor210) (FIG. 1C-D). Importantly, the inventors observed a significant association between ARID1A mutation and OS in CheckMate275 (N=139) and IMvigor210 (N=275) (FIG. 1E-F). In the CheckMate275, the inventors noted 11.4 months [0.95 CI: 5.48-NA] of median OS in patients with ARID1A mutation while median OS was 6.0 months [0.95 CI: 4.6-11.3] in patients with no ARID1A mutation, demonstrating significant association of ARID1A mutation and OS (p=0.03) (FIG. 1E). In the IMvigor 210 cohort, the inventors noted 15.4 months [0.95 CI: 9.2-NA] of median OS in patients with ARID1A mutation while median OS was 8.2 months [0.95 CI: 6.7-10.9] in patients with no ARID1A mutation, demonstrating significant association of ARID1A mutation and OS (p=0.03) (FIG. 1F). Of note, analysis of most mutated genes in the IMvigor210 cohort showed that genomic mutation of ARID1A was one of the two significant genes that was associated with OS (FIG. 7). Together, the inventors identified genomic mutation of ARID1A to be predictive of favorable responses to ICT in patients with mUCC.

### 2. Expression of CXCL13 in the Baseline Tumor Tissues Correlated with Improved Overall Survival

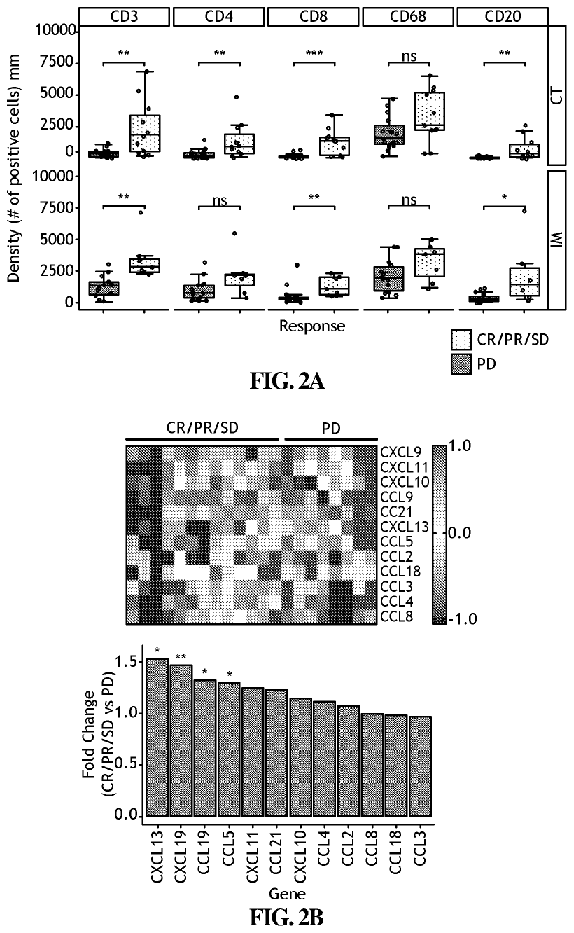

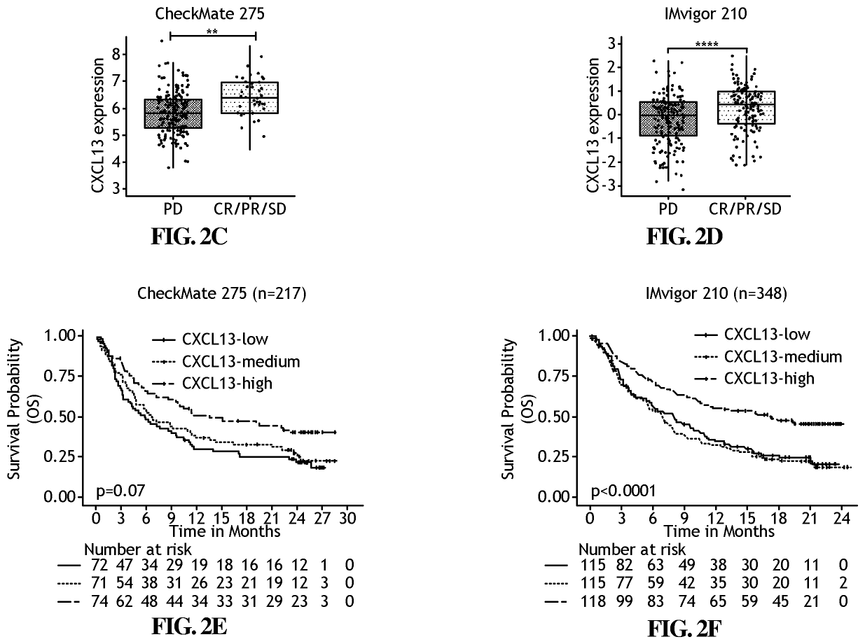

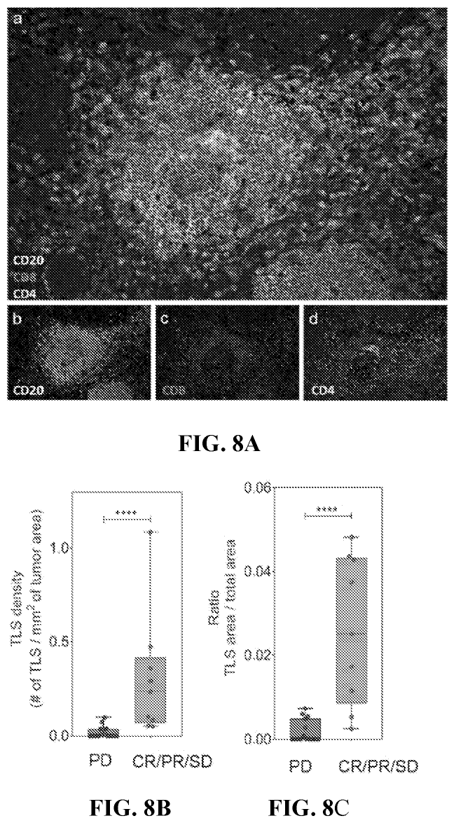

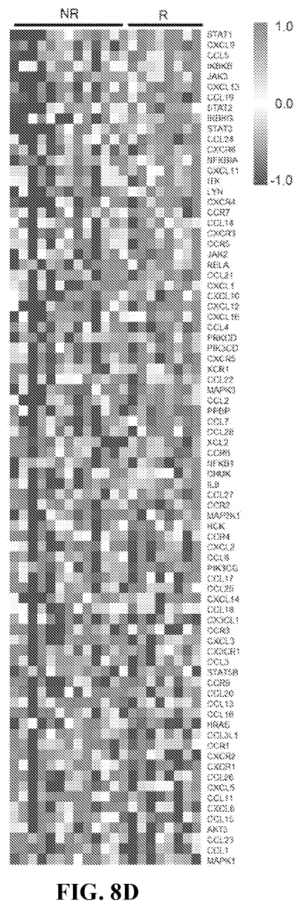

To integrate the genomic and immunological determinants of clinical responses to ICT in mUCC, the inventors sought to determine immunological biomarker that can predict response to ICT. To profile the immune sub-populations, the inventors performed immunohistochemistry (IHC) analysis on the baseline bladder tumor samples in the discovery cohort (N=31). The inventors noted predominant intra-tumoral infiltration of CD4+, CD8+T lymphocytes and CD20+B cells in responders (CR/PR/SD=11) compared to non-responders (PD=20) (FIG. 2A). The inventors also noted an increased density of tertiary lymphoid structures (TLS) in the baseline tumor tissues of responders compared to non-responders in patients with mUCC (FIG. 8A-C). To get a deeper insight into the plausible mechanism of immune infiltration and TLS formation, the inventors performed transcriptional analysis of baseline bladder tumor specimens from the discovery cohort (N=21, R=8 and NR=13) using targeted gene expression profiling (GEP) via a custom 739-gene NanoString panel. Interestingly, chemokine genes implicated in TLS formation (18) were found to be upregulated in baseline tumor samples from patients who responded to ICT (FIG. 2B and FIG. 8D). Specifically, CXCL13, CXCL9, CCL19 and CCL5 showed a significant differences between responders versus non-responders. While CXCL9, CCL19, and CCL5 play a role in T cell infiltration, CXCL13 plays a critical role in both T and B cell infiltration as well as TLS formation. Therefore, the inventors sought to determine the significance of CXCL13 in urothelial carcinoma in the context of ICT.

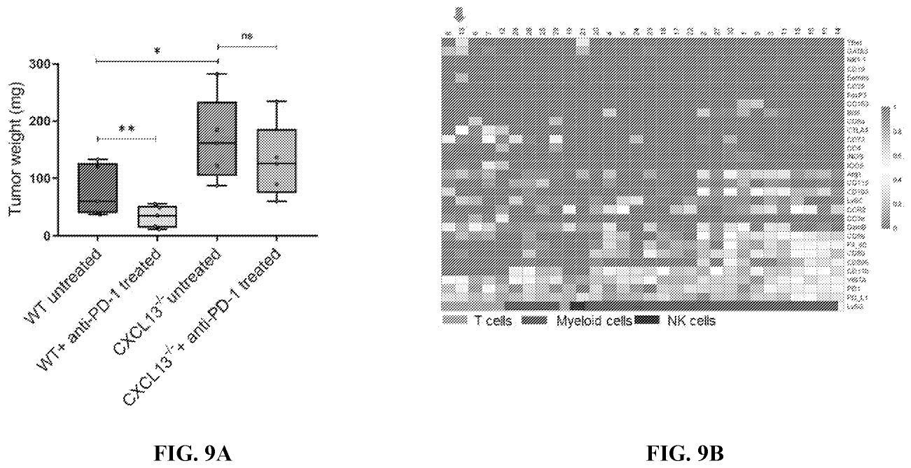

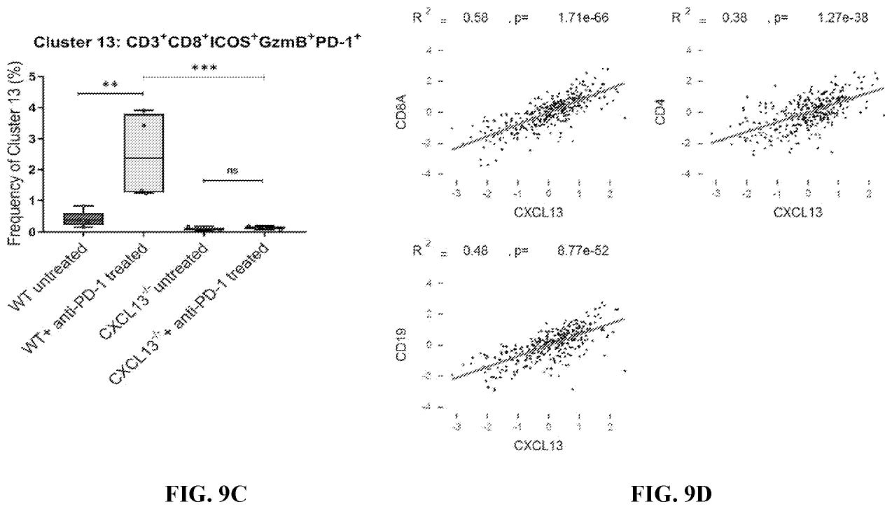

To test the role of CXCL13 in ICT mediated anti-tumor immunity in bladder cancer, the inventors used CXCL13 null (CXCL13^{-/-}) mice bearing murine bladder tumors (MB49). The inventors treated WT and CXCL13^{-/-} MB49 tumor bearing mice with anti-PD-1 therapy. While WT-MB49 tumor bearing mice responded to anti-PD-1 therapy, CXCL13^{-/-} mice did not respond to anti-PD-1 treatment (FIG. 9A). CyTOF analysis on the tumor infiltrating immune cells indicated that the abundance of intra-tumoral CD8⁺ICOS⁺GzmB⁺PD-1⁺ T cells were lower in the CXCL13^{-/-} mice compared to WT MB49 tumor bearing mice. Further, the subset of this activated CD8⁺ T cells failed to expand in response to anti-PD-1 treatment in CXCL13^{-/-} mice, as seen in their WT counterparts (FIG. 9B-C). This data suggested that absence of CXCL13 impeded activation and expansion of CD8⁺ T cells required for anti-PD-1 mediated anti-tumor immunity. Altogether, the murine studies have provided the evidence of direct association of CXCL13 with anti-PD-1 mediated anti-tumor immunity.

Next, the inventors tested the significance of CXCL13 as an immunological biomarker in the confirmatory cohorts (CheckMate 275 and IMvigor210). The inventors saw higher expression of CXCL13 in the responder (CR/PR/SD) group compared to non-responder (PD) both in CheckMate 275 and IMvigor210 cohort (FIG. 2C-D). Similar to the discovery cohort, the inventors noted a correlation of CXCL13 expression with CD4, CD8, and CD19 (FIG. 9D). Next, the inventors divided the cohort of bladder cancer patients, using 33^{th} and 66^{th} percentiles of CXCL13 expression as cutoffs for visualization. The inventors observed a significant association between CXCL13 levels and OS (p=0.007) in CheckMate 275 cohort (FIG. 2E). The inventors noted 13.5 months [0.95 CI: 7.22-NA] median OS in the CXCL13-high group, 6.6 months [0.95 CI: 4.6-11.4] in the CXCL13-medium and 5.7 [0.95 CI: 3.15-9.49] months median OS in the CXCL13-low groups, respectively. The inventors also noted a significant association between CXCL13 levels and OS (p<0.0001) in IMvigor 210 (FIG. 2F). The inventors noted 17.1 months [0.95 CI: 10.9-NA] median OS in the CXCL13-high group, 6.7 months [0.95 CI: 5.5-8.8] in the CXCL13-medium and 8.0 [0.95 CI: 5.8-10.8] months median OS in the CXCL13-low groups, respectively. Taken together, the inventors identified baseline CXCL13 expression in the tumor tissue to be predictive of favorable responses to ICT in patients with mUCC.

### 3. Combination of ARID1A Mutation and Baseline Expression of CXCL13 in the Tumor Tissue Demonstrate Synergistic Predictive Performance to ICT

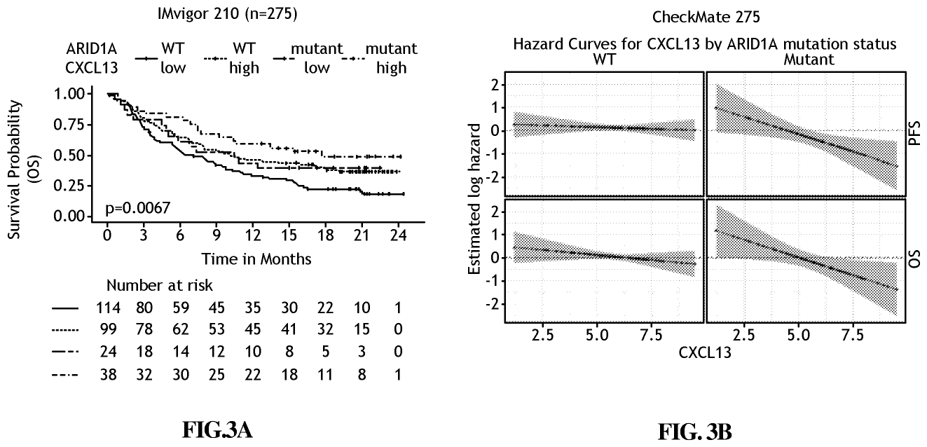

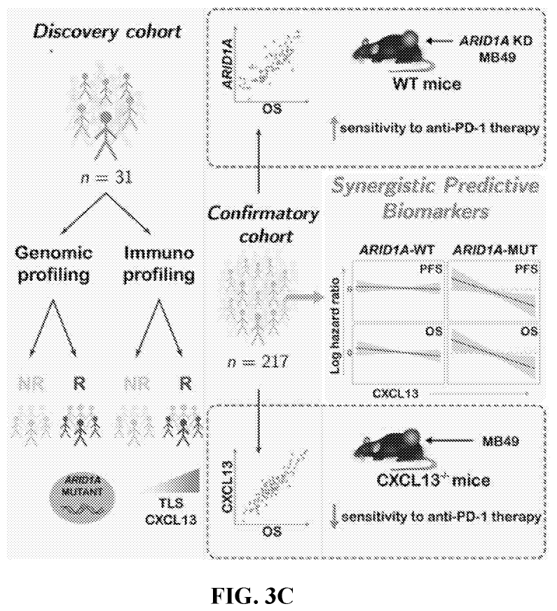

In the confirmatory cohorts, both ARID1A mutation and higher CXCL13 expression in the baseline tumor samples are associated with improved OS. Further, reverse translational studies provided evidence of direct association of CXCL13 expression and ARID1A mutation with anti-PD-1 mediated anti-tumor immunity. However, based on previous biomarker studies, the inventors have learned that single genomic or immunological biomarker fail to capture the entire spectrum of the tumor immune microenvironment. Therefore, recognizing the importance of both biomarkers, the inventors interrogated CXCL13 expression and ARID1A mutation for synergistic performance in predicting response to ICT in CheckMate275 and IMvigor210. The inventors noted that the combination of both the biomarkers in baseline tumor tissues correlated with improved OS compared to a single biomarker. Analysis of the patients in IMvigor 210, the inventors noted 17.8 months [0.95 CI: 10.4-NA] of median OS in patients with ARID1A mutation and CXCL13-high group, 10.5 months [0.95 CI: 5.1-NA] of median OS in patients with ARID1A mutation and CXCL13-low group, 10.2 months [0.95 CI: 7.9-19.1] in patients with no ARID1A mutation and CXCL13-high group, and 7.1 months [0.95 CI: 5.5-9.9] in patients with no ARID1A mutation and CXCL13-low group (FIG. 3A). Further, analysis of the hazard curves for CXCL13 by ARID1A mutation status demonstrated that higher CXCL13 expression have improved OS and PFS in CheckMate275. The association between CXCL13 expression and response was stronger in patients harboring ARID1A mutation compared to patients without ARID1A mutation (FIG. 3B). Here, the inventors showed that genomic mutation of ARID1A and baseline CXCL13 expression in the tumor tissues as two clinically relevant biomarkers in patients with mUCC receiving ICT that demonstrated synergistic predictive performance in predicting responses to ICT in patients with mUCC (FIG. 3C). This data highlights the fact that combination biomarker approaches are more important than single biomarkers in predicting clinical responses.

C. Discussion

It is increasingly clear that immune response to ICT is influenced by tumor as well as immune cells. Composite biomarkers through interrogation of tumor-immune ecosystem are needed to guide the treatment decisions. A recent report showed that patients with high TMB plus high T cell gene expression profile (GEP) have better response to ICT than TMB-hi or GEP-hi signature alone (21). The gene expression profile was calculated using an 18 gene signature (CCL5, CD27, CD274, CD276, CD8A, CMKLR1, CXCL9, CXCR6, HLA-DQA1, HLA-DRB1, HLA-E, IDO1, LAGS, NKG7, PDCD1, LG2, PSMB10, STAT1, and TIGIT). Although GEP and TMB has been correlated with response to ICT and improved progression free survival, it will need optimization in terms of GEP calculations in individual patients to incorporate into clinical practice. Further, unlike non-small cell lung cancer (NSCLC), the inventors do not have a tested cut-off for TMB in urothelial cancer, which could be used for the analysis as an established reference for patient selection. Comparatively, identification of single gene mutation, as well as baseline expression of single gene through currently available methods have immediate translational potential.

The data in this example showed that genomic mutation of ARID1A and baseline CXCL13 expression in the tumor tissues as two clinically relevant biomarkers in patients with mUCC receiving ICT that demonstrated synergistic predictive performance in predicting responses to ICT in patients with mUCC. Cancer Genome Atlas Project identified ARID1A as one of the recurrent mutations in patients with mUCC underlining the importance to evaluate the impact of ARID1A mutation to ICT. It was estimated that around 25% patients with mUCC carry ARID1A mutation (22). Similar to previous reports, the inventors noted that patients harboring ARID1A mutations have higher TMB compared to patients with the mutation. Further, patients with ARID1A mutation also have lower expression TGF□1. In the IMvigor210 trial, it was shown that TGF□1 can attenuate the response to anti-PD-L1 therapy by excluding the T cells from the tumor immune microenvironment. The inventors did not note any difference in CXCL13 expression between ARID1A-WT and ARID1A-mutant patients suggesting ARID1A mutation and expression of CXCL13 might not be directly linked, rather could work synergistically in modulating the tumor immune microenvironment. Altogether, this study provided insight into two biomarkers in predicting responses to ICT in patients with mUCC and highlighted the utility of composite biomarkers integrating tumor and immune microenvironment, in the selection of patient cohorts to receive ICT. The inventors will test this concept prospectively in future clinical trials.

D. Methods

1. Patients and samples

Metastatic urothelial carcinoma discovery cohort: samples were collected as a part of 1) phase 2 study assessing the safety and efficacy of nivolumab in metastatic or surgically unresectable urothelial carcinoma whose disease progressed or recurred despite previous treatment with at least one platinum-based chemotherapy regimen/NCT02387996 and 2) phase 1/2, open-label study of nivolumab monotherapy or nivolumab combined with ipilimumab in subjects with advanced or metastatic solid tumors/NCT01928394. Patient samples were collected after appropriate informed consent was obtained on MD Anderson IRB-approved protocol PA13-0291.

For the validation cohort, the inventors used The CheckMate 275 (NCT02387996) dataset comprising of 270 patients with platinum-resistant mUCC treated with nivolumab on a phase II clinical trial that has been described previously (14,19). The response assessments and survival follow-up of this cohort has previously been described (14); the Objective Responses were determined based on a blinded independent review committee assessment. Archival formalin-fixed paraffin embedded mUCC tumor specimens were submitted for each patient prior to initiation of nivolumab. Gene expression was measured using the HTG EdgeSeq system (HTG Molecular, Tuczon, AZ) Oncology and Immuno-Oncology Biomarker Panels. Data were transformed into log 2 Trimmed mean of M-values (TMM), normalized counts per million (CPM) prior to analysis based on manufacturer's instructions.

2. Mice

C57BL/6 (5-7 weeks) mice were purchased from the National Cancer Institute (Frederick, Md.) and CXCL13 null (CXCL13^{-/-}) mice in the C57BL/6 background (stock no. 005626, 5-7 weeks) were purchased from The Jackson Laboratory (Bar Harbor, Me.). All mice were kept in specific pathogen-free conditions in the Animal Resource Center at The University of Texas MD Anderson Cancer Center. Animal protocols were approved by the Institutional Animal Care and Use Committee (IACUC) of The University of Texas MD Anderson Cancer Center.

3. Cell Lines and Tumor Model

Murine bladder cancer cell line (MB49) were provided by Dr. A. Kamat (The University of Texas MD Anderson Cancer Center, Houston, Tex.). 2×10⁵ (MB49) cells were injected subcutaneously, in the right flank of C57BL/6 mice (5 to 10 mice per group).

4. Whole Exome Sequencing Data Analysis

DNA from FFPE tissues and peripheral blood was obtained using the QiaAmp DNA FFPE Tissue Kit and QiaAmp DNA Mini kit, respectively (QiaGen). Whole-exome sequencing experiments were performed on tumor tissues from 68 patients (37 responders and 31 non-responders). Normal peripheral blood was used as control. Genomic DNA (250 ng) was sheared using low Tris-EDTA buffer. KAPA Hyper Prep Kit (#KK8504) was used for end repair, A-base addition, adaptor ligation and library enrichment PCR. Library construction was performed following the manufacturer's instructions. Sample concentrations were measured following library construction using the Agilent Bioanalyzer. Hybridization reaction was then performed per the manufacturer's instructions. In terms of target capture, the Agilent SureSelect-XT Target Enrichment (#5190-8646) protocol was followed, according to the manufacturer's guidelines. The libraries were then normalized to equal concentrations using an Eppendorf Mastercycler EP Gradient instrument and pooled to equimolar amounts on the Agilent Bravo B platform. Libraries were quantified using the KAPA Library Quantification Kit (#KK4824). The BWA aligner (bwa-0.7.5a) was applied to map the raw reads to the human hg19 reference genome (UCSC genome browser: genome.ucsc.edu). The average exome-wide coverage ranges in 75.6-294.9 fold (median 182.6) in tumor samples and 47.2-140.4 fold (median 91.56) in the matched normal samples. The Picard (v1.112, http://broadinstitute.github.io/picard/) module “MarkDuplicates” was applied to mark the duplicate reads. Then the “IndelRealigner” and “BaseRecalibrator” modules of the Genome Analysis Toolkit were applied to perform indel realignment and base quality recalibration. MuTect (v1.1.4) and Pindel (v0.2.4t) were applied to each tumor and matched normal PBMC sample to detect somatic single nucleotide variants (SNVs) and small insertions/deletions. To ensure specificity, the following criteria was applied to filter the detected somatic SNVs and indels: the average is at least 20 reads for the tumor and 10 for the normal; the total number of reads supporting the variant is at least 4 and the tumor alternate allele frequency (AF) is at least 5%; the MuTect LOD score is at least 9.0. The allele frequency from the normal sample is less than 0.01%. To avoid including germline mutations, the inventors further required the AF<0.01 from the ESP6500, 1000 genome and EXAC databases. If repetitive sequences were detected within 25-bp in the downstream regions of an indel, that indel was discarded. Only missense, nonsense and frame-shifting indels in the exonic regions and mutations at splice sites and UTR regions were included in the downstream analysis. There are 24 samples that passed the QC steps with meaningful overall response calls: 11 from responders (R) and 13 from non responders (NR). The filtered mutation data were analyzed using the R package maftools (23).

5. Mass cytometry (CyTOF)

Freshly collected MB49 tumors from the mice were dissociated with Liberase/DNAse solution, incubated for 30 minutes at 37° C. following which single cell suspensions were made. Around 3 million cells per sample was taken for CyTOF staining. Antibodies were either purchased pre-conjugated from Fluidigm or purchased purified and conjugated in house using MaxPar X8 Polymer kits (Fluidigm) according to the manufacturer's instructions. Briefly, samples were stained with cell-surface antibodies for 30 minutes at 4° C. and then stained for viability with 5 μM cisplatin in 5% FACS buffer for 1 min at RT. Samples were then washed, fixed and permeabilized (eBioscience) and stained with intracellular antibodies for 30 mins at 4° C. Post staining, the samples were washed and barcoded using the manufacturer's protocol (Fluidigm) after which they were incubated with 125 μM Jr intercalator (Fluidigm) in 1.6% PFA/PBS and stored at 4° C. overnight. The cells were then washed with PBS the next day and stored until acquisition. Right before acquisition, samples were washed twice with Milli-Q water, resuspended in water containing EQ 4 element beads (Fluidigm) and run on a Helios mass cytometer (Fluidigm).

6. Mass Cytometry Analysis

Files were manually gated in FlowJo by event length for singlets, live/dead discrimination and using CD45 lineage marker for immune cells. Fcs files were then loaded into R using the flowCore package as a flowset for downstream analysis. Data were arcsinh transformed using a coefficient of 5 (x_transformed=asinh(x/5)). Clustering analysis was performed using the FlowSOM and the ConsensusClusterPlus packages as previously described ²⁴. Clusters were identified using PhenoGraph on a per sample basis in the space formed by these principal components and cluster frequency and t-SNE plots were generated.

7. Nanostring

RNA were isolated from formalin fixed paraffin embedded (FFPE) tumor sections by de-waxing using deparaffinization solution (Qiagen, Valencia, Calif.), and total RNA was extracted using the RecoverALL™ Total Nucleic Acid Isolation kit (Ambion, Austin, Tex.) according to the manufacturer's instructions. The RNA purity was assessed on the ND-Nanodrop1000 spectrometer (Thermo Scientific, Wilmington, Mass., USA). For the NanoString platform, 100 ng of RNA was used to detect immune gene expression using nCounter PanCancer Immune Profiling panel along with custom CodeSet. Counts of the reporter probes were tabulated for each sample by the nCounter Digital Analyzer and raw data output was imported into nSolver (http://www.nanostring.com/products/nSolver). nSolver data analysis package was used for normalization and hierarchical clustering heatmap analysis were performed with Qlucore Omics Explorer version 3.5 software (Qlucore, NY, USA).

8. Immunohistochemistry

Hematoxylin and Eosin (H&E) and immunohistochemistry (IHC) staining was performed on FFPE tumor tissue sections. The tumor tissues were fixed in 10% formalin, embedded in paraffin, and transversely sectioned. 4 μm sections were used for the histo-pathological studies. Sections were stained with mouse or rabbit anti-human monoclonal antibodies against CD3 (Dako, Cat#A0452), CD4 (Novocastra, CD4-368-L-A), CD8 (Thermo Scientific, MS-457-S), CD45RO (Novocastra, PA0146), GzmB (Leica Microsystems, PA0291), CD68 (Dako, M0876), and CD20 (DAKO, M0755). All sections were counterstained with H&E, dehydrated and mounted. All sections were processed with peroxidase-conjugated avidin/biotin and 3′-3-diaminobenzidine (DAB) substrate (Leica Microsystem) and slides were scanned and digitalized using the scanscope system from Scanscope XT, Aperio/Leica Technologies. Quantitative analysis of IHC staining was conducted using the image analysis software ImageScope-Aperio/Leica. Five random areas (1 mm² each) were selected using a customized algorithm for each marker in order to determine the number of positive cells at high power field (HPF). The data is expressed as a density (total number of positive cells/mm² area). IHC staining was interpreted in conjunction with H&E stained sections.

9. Multiplex Immunofluorescence Assay and Analysis

For multiplex IF staining, the Opal protocol staining method (Stack et al., 2014) was used and the slides were stained analyzed for the following markers: CD20 (Dako, cat# M0755) with subsequent visualization using fluorescein Cy5; CD4 (CM153BK, Biocare) with subsequent visualization using fluorescein Cy3; CD8 (M7103, Dako) with subsequent visualization using fluorescein Cy5.5 and nuclei visualized with DAPI. All of the sections were mounted using Vectashield Hardset 895 mounting media.

Slides were scanned using the Vectra slide scanner (PerkinElmer). For each marker, the mean fluorescent intensity per case was then determined as a base point from which positive calls could be established. For multispectral analysis, each of the individually stained section was utilized to establish the spectral library of the fluorophores. Five random areas on each sample were analyzed blindly by a pathologist at 20× magnification.

10. Tertiary Lymphoid Structure Quantification

Tertiary Lymphoid Structures (TLS) were qualified and quantified using both H&E and CD20 IHC staining. Structures were identified as aggregates of lymphocytes having histologic features with analogous structures to that of lymphoid tissue with follicles, appearing in the tumor area. For the current study, criteria used for the quantification of TLS includes: 1) the total number of structures identified either within the tumoral area or in direct contact with the tumoral cells on the margin of the tumors (numbers of TLS/1 mm² area); and 2) a normalization of the total area occupied by the TLSs in relation of the total area of the tumor analyzed (ratio: area of TLS/area tumor+TLSs).

11. Statistics

Cox Proportional Hazards (PH) regression models were used to assess the dependence of PFS or OS on gene expression score. Proportional hazards assumptions were assessed by examination of scaled Schoenfeld residuals. For all Cox PH models, the PH assumption appeared reasonable. The magnitudes of associations were summarized by Hazard Ratios (HRs). Reported HRs were scaled to z-score of the biomarker scores. Because the effects of the biomarkers were constrained to be linear in these models, the HR estimates depended only standard deviation of the gene expression score, not on the individual values. Two-sided 95% confidence intervals for HRs were based on Wald test statistics. Kaplan-Meier plots based on categorization of the biomarker scores were used to illustrate associations with PFS and OS. The magnitudes of associations were summarized by odds ratios (ORs), scaled in the same way as the reported HRs. Two-sided 95% confidence intervals for ORs were based on Wald test statistics. Two-sided 95% confidence intervals for objective response rate were estimated by the Clopper-Pearson exact method. Likelihood-ratio tests were used to test overall biomarker effects. Kaplan-Meier plots based on categorization of the biomarker scores were used to illustrate associations with PFS or OS. All data analyses were performed with R 3.4.1 for Linux and R 3.5.2 for Windows.

For murine experiments, all data are representative of at least two to three independent experiments with 5-10 mice in each in vivo experiment. The data are expressed as mean±standard error of the mean (SEM) and were analyzed using Prism 7.0 statistical analysis software (GraphPad Software, La Jolla, Calif.). Student t-tests (two tailed), ANOVA, and Bonferroni multiple comparison tests were used to identify significant differences (p<0.05) between treatment groups.

### * * *

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and the publications referred to throughout the specification, to the extent that they provide exemplary procedural or other details supplementary to those set forth herein, are specifically incorporated herein by reference.
1. Siegel, R. L., Miller, K. D. & Jemal, A. Cancer Statistics, 2017. CA Cancer J Clin 67, 7-30 (2017).
2. Powles, T., et al. MPDL3280A (anti-PD-L1) treatment leads to clinical activity in metastatic bladder cancer. Nature 515, 558-562 (2014).
3. Rosenberg, J. E., et al. Atezolizumab in patients with locally advanced and metastatic urothelial carcinoma who have progressed following treatment with platinum-based chemotherapy: a single-arm, multicentre, phase 2 trial. Lancet 387, 1909-1920 (2016).
4. Balar, A. V., et al. Atezolizumab as first-line treatment in cisplatin-ineligible patients with locally advanced and metastatic urothelial carcinoma: a single-arm, multicentre, phase 2 trial. Lancet 389, 67-76 (2017).
5. Sharma, P., et al. Nivolumab in metastatic urothelial carcinoma after platinum therapy (CheckMate 275): a multicentre, single-arm, phase 2 trial. The Lancet. Oncology 18, 312-322 (2017).
6. Bellmunt, J., et al. Pembrolizumab as Second-Line Therapy for Advanced Urothelial Carcinoma. N Engl J Med 376, 1015-1026 (2017).
7. Massard, C., et al. Safety and Efficacy of Durvalumab (MEDI4736), an Anti-Programmed Cell Death Ligand-1 Immune Checkpoint Inhibitor, in Patients With Advanced Urothelial Bladder Cancer. J Clin Oncol 34, 3119-3125 (2016).
8. Drake, C. G., Bivalacqua, T. J. & Hahn, N. M. Programmed Cell Death Ligand-1 Blockade in Urothelial Bladder Cancer: To Select or Not to Select. J Clin Oncol 34, 3115-3116 (2016).
9. Apolo, A. B., et al. Avelumab, an Anti-Programmed Death-Ligand 1 Antibody, In Patients With Refractory Metastatic Urothelial Carcinoma: Results From a Multicenter, Phase Ib Study. J Clin Oncol 35, 2117-2124 (2017).
10. Gopalakrishnan, D., Koshkin, V. S., Ornstein, M. C., Papatsoris, A. & Grivas, P. Immune checkpoint inhibitors in urothelial cancer: recent updates and future outlook. Ther Clin Risk Manag 14, 1019-1040 (2018).
11. Aggen, D. H. & Drake, C. G. Biomarkers for immunotherapy in bladder cancer: a moving target. J Immunother Cancer 5, 94 (2017).
12. Sharma, P. & Allison, J. P. The future of immune checkpoint therapy. Science 348, 56-61 (2015).
13. Sharma, P. Immune Checkpoint Therapy and the Search for Predictive Biomarkers. Cancer J 22, 68-72 (2016).
14. Sharma, P., et al. Nivolumab monotherapy in recurrent metastatic urothelial carcinoma (CheckMate 032): a multicentre, open-label, two-stage, multi-arm, phase 1/2 trial. The Lancet. Oncology 17, 1590-1598 (2016).
15. Mariathasan, S., et al. TGFbeta attenuates tumour response to PD-L1 blockade by contributing to exclusion of T cells. Nature 554, 544-548 (2018).
16. Wang, X., et al. Expression of p270 (ARID1A), a component of human SWI/SNF complexes, in human tumors. Int J Cancer 112, 636 (2004).
17. Shen, J., et al. ARID1A deficiency promotes mutability and potentiates therapeutic antitumor immunity unleashed by immune checkpoint blockade. Nature medicine 24, 556-562 (2018).
18. Messina, J. L., et al. 12-Chemokine gene signature identifies lymph node-like structures in melanoma: potential for patient selection for immunotherapy? Scientific reports 2, 765 (2012).
19. Wang, L., et al. EMT- and stroma-related gene expression and resistance to PD-1 blockade in urothelial cancer. Nat Commun 9, 3503 (2018).
20. Powles, T., et al. Clinical efficacy and biomarker analysis of neoadjuvant atezolizumab in operable urothelial carcinoma in the ABACUS trial. Nature medicine 25, 1706-1714 (2019).
21. Cristescu, R., et al. Pan-tumor genomic biomarkers for PD-1 checkpoint blockade-based immunotherapy. Science 362(2018).
22. Cancer Genome Atlas Research, N. Comprehensive molecular characterization of urothelial bladder carcinoma. Nature 507, 315-322 (2014).
23. Mayakonda, A., Lin, D. C., Assenov, Y., Plass, C. & Koeffler, H. P. Maftools: efficient and comprehensive analysis of somatic variants in cancer. Genome Res 28, 1747-1756 (2018).
24. Nowicka, M., et al. CyTOF workflow: differential discovery in high-throughput high-dimensional cytometry datasets. F1000Res 6, 748 (2017).

What is claimed is:

1. A method of treating cancer in a subject comprising administering to the subject immune checkpoint blockade (ICB) therapy after the subject has been determined to have increased expression of CXCL13 in a biological sample from the subject.

2. The method of claim 1, wherein the cancer comprises bladder cancer.

3. The method of claim 2, wherein the bladder cancer comprises urothelial cancer.

4. The method of any one of claims 1-3, wherein the subject has been diagnosed with cancer.

5. The method of any one of claims 1-4, wherein the bladder cancer is further defined as bladder cancer with high-density tertiary lymphoid structure.

6. The method of claim 1 or 3, wherein the cancer comprises cholangiocarcinoma and/or a cancer of the biliary tract.

7. The method of any one of claims 1-6, wherein the subject is further defined as one that is ARID1A mutant.

8. The method of claim 7, wherein the ARID1A mutant subject is one that has harbors a mutation in at least one allele of the ARID1A gene that results in at least partial loss of protein expression or function.

9. The method of claim 7 or 8, wherein the expression level of CXCL13 and the ARID1A mutation status was determined in the subject and wherein the ARID1A mutation status was determined prior to the CXCL13 expression level.

10. The method of claim 7 or 8, wherein the expression level of CXCL13 and the ARID1A mutation status was determined in the subject and wherein the CXCL13 expression level was determined prior to the ARID1A mutation status.

11. The method of any one of claims 1-10, wherein the subject has been determined to have decreased expression of ARID1A.

12. The method of any one of claims 1-11, wherein the subject has been determined to have increased expression of CXCL13 and/or decreased expression of ARID1A by detecting or measuring mRNA expression or protein expression of CXCL13 and/or ARID1A.

13. The method of any one of claims 1-12, wherein the expression level and/or mutation status of CXCL13 and/or ARID1A was determined by NanoString analysis.

14. The method of any one of claims 1-13, wherein the subject has been determined to have increased expression of CXCL13 by performing an immunoassay on the biological sample.

15. The method of claim 14, wherein the immunoassay comprises immunohistochemistry.

16. The method of any one of claims 1-13, wherein the method further comprises measuring the expression level of CXCL13 and/or ARID 1A in a biological sample from the subject.

17. The method of claim 16, wherein measuring the expression level comprises detecting or measuring mRNA expression or protein expression.

18. The method of any one of claims 1-17, wherein the expression of CXCL13 in increased or was determined to be increased, as compared to a control.

19. The method of any one of claims 1-18, wherein the cancer comprises recurrent cancer.

20. The method of any one of claims 1-19, wherein the subject has been previously treated for the cancer with an anticancer agent.

21. The method of claim 20, wherein the previous treatment comprises a chemotherapeutic.

22. The method of claim 21, wherein the chemotherapeutic comprises at least one platinum-based chemotherapeutic agent.

23. The method of any one of claims 20-22, wherein the subject has been determined to be non-responsive to the previous therapy.

24. The method of any one of claims 1-22, wherein the biological sample comprises a tumor sample or a biopsy sample.

25. The method of claim 24, wherein the biological sample comprises a biopsy comprising tumor cells and/or components of the tumor micro environment.

26. The method of any one of claims 1-25, wherein the ICB therapy comprises a monotherapy or a combination ICB therapy.

27. The method of any one of claims 1-26, wherein the ICB therapy comprises an inhibitor of PD-1, PDL1, PDL2, CTLA-4, B7-1, and/or B7-2.

28. The method of any one of claims 1-27, wherein the ICB therapy comprises an anti-PD-1 monoclonal antibody and/or an anti-CTLA-4 monoclonal antibody.

29. The method of claim 28, wherein the ICB therapy comprises one or more of nivolumab, pembrolizumab, pidilizumab, ipilimumab, and tremelimumab.

30. The method of claim 28, wherein the ICB therapy comprises nivolumab monotherapy.

31. The method of claim 28, wherein the ICB therapy comprises nivolumab and ipilimumab combination therapy.

32. The method of any one of claims 1-27, wherein the method further comprises administering at least one additional anticancer treatment.

33. The method of claim 32, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

34. The method of any one of claims 4-33, wherein the control comprises a cut-off value or a normalized value.

35. The method of any one of claims 1-34, wherein the increased expression level comprises a level of expression or normalized level of expression that is determined to be increased as compared to a control.

36. The method of claim 35, wherein the control represents an expression level of CXCL13 in a biological sample from a subject that has been determined to not respond to ICB therapy.

37. The method of any one of claims 1-34, wherein the subject was determined to have a level of CXCL13 expression that was not significantly different than a control or within one standard deviation from a control, wherein the control represents an expression level of CXCL13 in a biological sample from a subject that has been determined to respond to ICB therapy.

38. A method for predicting a response to ICB therapy in a subject having cancer, the method comprising:
(a) determining the expression level of CXCL13 in a sample from the subject;
(b) comparing the expression level of CXCL13 in a sample from the subject to a control; and
(c) predicting that the subject will respond to the ICB therapy after (i) an increased expression level of CXCL13 is detected in a biological sample from the subject as compared to a control, wherein the control represents an expression level of CXCL13 in a biological sample from a subject that has been determined to not respond to ICB therapy; or (ii) a non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to respond to ICB therapy; or
(d) predicting that the subject will not respond to the ICB therapy after (i) a decreased or non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to not respond to ICB therapy; or (ii) a decreased expression level of CXCL13 is detected in a biological sample from the subject as compared to a control, wherein the control represents an expression level of CXCL13 in a biological sample from a subject that has been determined to respond to ICB therapy.

39. The method of claim 38, wherein the cancer comprises bladder cancer.

40. The method of claim 39, wherein the bladder cancer comprises urothelial cancer.

41. The method of claim 38, wherein the cancer comprises cholangiocarcinoma and/or a cancer of the biliary tract.

42. The method of any one of claims 38-41, wherein the subject is further defined as one that is ARID1A mutant or is further defined as one that has reduced ARID1A expression.

43. The method of claim 42, wherein the ARID1A mutant subject is one that has harbors a mutation in at least one allele of the ARID1A gene that results in at least partial loss of protein expression or function.

44. The method any one of claims 38-40, wherein the method further comprises determining the expression level or mutation status of ARID1A in the subject.

45. The method of claim 44, wherein the expression level or mutation status of ARID1A was determined prior to the expression level of CXCL13.

46. The method of claim 44, wherein the expression level of CXCL13 was determined prior to the expression level or mutation status of ARID1A.

47. The method of any one of claims 42-46, wherein the subject is predicted to respond to ICB therapy when the expression level of ARID1A is reduced compared to a control or when ARID1A is determined to be mutant.

48. The method of any one of claims 42-46, wherein the subject is predicted to not respond when the expression level of ARID1A is not substantially different than a control level of expression and/or when ARID1A is determined to be wild-type.

49. The method of claim 47, wherein the mutant ARID1A comprises a mutation in at least one allele of the ARID1A gene that results in at least partial loss of protein expression or function.

50. The expression level of any one of claims 38-49, wherein the expression level of CXCL13 and/or ARID1A was measured or detected by detecting or measuring mRNA expression or protein expression of CXCL13 and/or ARID1A.

51. The method of any one of claims 38-50, wherein the expression level and/or mutation status of CXCL13 and/or ARID1A was measured or detected by NanoString analysis.

52. The method of any one of claims 38-51, wherein the expression level of CXCL13 was determined by an immunoassay.

53. The method of claim 52, wherein the expression level of CXCL13 was determined by immunohistochemistry.

54. The method of any one of claims 38-53, wherein the method further comprises treating the subject predicted to respond to ICB therapy with ICB therapy.

55. The method of any one of claims 38-54, wherein the biological sample comprises a tumor sample or a biopsy sample.

56. The method of any one of claims 38-55, wherein the ICB therapy comprises a monotherapy or a combination ICB therapy.

57. The method of any one of claims 38-56, wherein the ICB therapy comprises an inhibitor of PD-1, PDL1, PDL2, CTLA-4, B7-1, and/or B7-2.

58. The method of any one of claims 38-57, wherein the ICB therapy comprises an anti-PD-1 monoclonal antibody and/or an anti-CTLA-4 monoclonal antibody.

59. The method of claim 58, wherein the ICB therapy comprises one or more of nivolumab, pembrolizumab, pidilizumab, ipilimumab, and tremelimumab.

60. The method of claim 59, wherein the ICB therapy comprises nivolumab monotherapy.

61. The method of claim 59, wherein the ICB therapy comprises nivolumab and ipilimumab combination therapy.

62. The method of any one of claims 38-61, wherein the method further comprises administering at least one additional anticancer treatment.

63. The method of claim 62, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

64. The method of any one of claims 38-63, wherein the control comprises a cut-off value or a normalized value.

65. The method of any one of claims 38-64, wherein the expression level comprises a normalized level of expression.

66. A method comprising detecting CXCL13 in a biological sample from a subject with cancer.

67. The method of claim 66, wherein the cancer comprises bladder cancer.

68. The method of claim 67, wherein the bladder cancer comprises urothelial cancer.

69. The method of any one of claims 66-68, wherein the subject has been diagnosed with cancer.

70. The method of any one of claims 67-69, wherein the bladder cancer is further defined as bladder cancer with high-density tertiary lymphoid structure.

71. The method of claim 66 or 69, wherein the cancer comprises cholangiocarcinoma and/or a cancer of the biliary tract.

72. The method of any one of claims 66-71, wherein the subject is further defined as one that is ARID1A mutant or is further defined as one that has reduced ARID1A expression.

73. The method of claim 72, wherein the ARID1A mutant subject is one that has harbors a mutation in at least one allele of the ARID1A gene that results in at least partial loss of protein expression or function.

74. The method of any one of claims 66-73, wherein the method further comprises detecting the expression level or mutation status of ARID1A in the biological sample.

75. The method of any one of claims 72-74, wherein the expression level or mutation status of ARID1A was determined prior to the expression level of CXCL13.

76. The method of any one of claims 72-74, wherein the expression level of CXCL13 was determined prior to the expression level or mutation status of ARID1A.

77. The method of any one of claims 66-76, wherein the expression level of CXCL13 and/or ARID1A was measured or detected by measuring or detecting the mRNA expression or protein expression of CXCL13 and/or ARID1A.

78. The method of any one of claims 66-77, wherein the expression level and/or mutation status of CXCL13 and/or ARID1A was determined by NanoString analysis.

79. The method of any one of claims 66-78, wherein the method further comprises comparing the level of expression of CXCL13 and/or ARID 1A to a level of expression in a control.

80. The method of any one of claims 66-79, wherein the bladder cancer comprises urothelial cancer.

81. The method of any one of claims 66-80, wherein the biological samples comprises a tumor sample or a biopsy sample.

82. The method of any one of claims 66-81, wherein the control comprises a cut-off value or a normalized value.

83. The method of any one of claims 66-82, wherein the expression level comprises a normalized level of expression.

84. The method of any one of claims 66-83, wherein the CXCL13 expression is detected by an immunoassay.

85. The method of claim 84, wherein CXCL13 expression is detected by immunohistochemistry.

86. The method of any one of claims 66-85, wherein the subject has been determined to be a candidate for ICB therapy.

87. The method of any one of claims 66-86, wherein the subject is currently being treated with ICB therapy, has received at least one ICB therapy, or wherein the subject has not been treated with ICB therapy.

88. The method of anyone of claims 79-87, wherein the control comprises a biological sample from a subject that does not respond to ICB therapy.

89. The method of anyone of claims 79-87, wherein the control comprises a biological sample from a subject that responds to ICB therapy.

90. The method of claim 88 or 89 wherein the subject is determined to have a higher expression level than the control.

91. The method of claim 88 or 89 wherein the subject is determined to have a lower expression level than the control.

92. The method of claim 88 or 89 wherein the subject is determined to have a level of expression that is not significantly different than the control.

93. The method of any one of claims 86-92, wherein the ICB therapy comprises a monotherapy or combination ICB therapy.

94. The method of any one of claims 86-93, wherein the ICB therapy comprises an inhibitor of PD-1, PDL1, PDL2, CTLA-4, B7-1, and/or B7-2.

95. The method of any one of claims 86-94, wherein the ICB therapy comprises an anti-PD-1 monoclonal antibody and/or an anti-CTLA-4 monoclonal antibody.

96. The method of claim 95, wherein the ICB therapy comprises one or more of nivolumab, pembrolizumab, pidilizumab, ipilimumab, and tremelimumab.

97. The method of claim 96, wherein the ICB therapy comprises nivolumab monotherapy.

98. The method of claim 96, wherein the ICB therapy comprises nivolumab and ipilimumab combination therapy.

99. A method of treating cancer in a subject comprising administering to the subject immune checkpoint blockade (ICB) therapy after the subject has been determined to have increased expression of CXCL13 in a biological sample from the subject; wherein the subject is one that harbors a mutation in at least one allele of the ARID1A gene that results in at least partial loss of protein expression or function.

100. The method of claim 99, wherein the subject was determined to a mutation in at least one allele of the ARID1A gene by NanoString analysis.

101. The method of claim 99 or 100, wherein the subject has been determined to have increased expression of CXCL13 by performing immunohistochemistry to detect CXCL13 expression in the biological sample from the subject.

102. A method for predicting a response to ICB therapy in a subject having cancer and having a mutation in at least one allele of the ARID1A gene that results in at least partial loss of protein expression or function, the method comprising:
(a) determining the expression level of CXCL13 in a sample from the subject;
(b) comparing the expression level of CXCL13 in a sample from the subject to a control; and
(c) predicting that the subject will respond to the ICB therapy after (i) an increased expression level of CXCL13 is detected in a biological sample from the subject as compared to a control, wherein the control represents an expression level of CXCL13 in a biological sample from a subject that has been determined to not respond to ICB therapy; or (ii) a non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to respond to ICB therapy; or
(d) predicting that the subject will not respond to the ICB therapy after (i) a decreased or non-significantly different expression level of CXCL13 is detected in a biological sample from the subject as compared to a control or an expression level of CXCL13 that is within one standard deviation to a control is detected in a biological sample from the subject, wherein the control represents an expression level of CXCL13 in a biological sample from a subject that has been determined to not respond to ICB therapy; or (ii) a decreased expression level of CXCL13 is detected in a biological sample from the subject as compared to a control, wherein the control represents an expression level of CXCL13 in a biological sample from a subject that has been determined to respond to ICB therapy.

103. The method of claim 102, wherein the subject was determined to a mutation in at least one allele of the ARID1A gene by NanoString analysis.

104. The method of claim 102 or 103, wherein the expression level of CXCL13 was determined by performing immunohistochemistry to detect CXCL13 expression in the biological sample from the subject.

105. A method comprising detecting CXCL13 in a biological sample from a subject with cancer;
wherein the subject is one that harbors a mutation in at least one allele of the ARID1A gene that results in at least partial loss of protein expression or function.

106. The method of claim 105, wherein the subject was determined to a mutation in at least one allele of the ARID1A gene by NanoString analysis.

107. The method of claim 105 or 106, wherein the expression level of CXCL13 is detected by performing immunohistochemistry to detect CXCL13 expression in the biological sample from the subject.
